SEQUENCE LISTING (22 sequences)

SEQ ID NO: 1 - protein, 222 aa, Homo sapiens
VPCVSGGLPKPANITFLSINMKNVLQWTPPEGLQGVKVTYTVQYFIYGQKKWLNKSECRNINRTYCDLSAETSDYEHQYYAKVKAIWGTKCSKWAESGRFYPFLETQIGPPEVALTTDEKSISVVLTAPEKWKRNPEDLPVSMQQIYSNLKYNVSVLNTKSNRTWSQCVTNHTLVLTWLEPNTLYCVHVESFVPGPPRRAQPSEKQCARTLKDQSSEFKAKI

SEQ ID NO: 2 - protein, 197 aa, Homo sapiens
VAILPAPQNLSVLSTNMKHLLMWSPVIAPGETVYYSVEYQGEYESLYTSHIWIPSSWCSLTEGPECDVTDDITATVPYNLRVRATLGSQTSAWSILKHPFNRNSTILTRPGMEITKDGFHLVIELEDLGPQFEFLVAYWRREPGAEEHVKMVRSGGIPVHLETMEPGAAYCVKAQTFVKAIGRYSAFSQTECVEVQG

SEQ ID NO: 3 - protein, 115 aa, Homo sapiens
AQIGPPEVALTTDEKSISVVLTAPEKWKRNPEDLPVSMQQIYSNLKYNVSVLNTKSNRTWSQCVTNHTLVLTWLEPNTLYCVHVESFVPGPPRRAQPSEKQCARTLKDQSSEFKA

SEQ ID NO: 4 - protein, 47 aa, Homo sapiens
VPCVSGGLPKPANITFLSINMKNVLQWTPPEGLQGVKVTYTVQYFIS

SEQ ID NO: 5 - protein, 47 aa, Homo sapiens
VPCVSGGLPKPANITFLSINMKNVLQWTPPEGLQGVKVTYTVQYFIT

SEQ ID NO: 6 - protein, 210 aa, Homo sapiens
PEDPSDLLQHVKFQSSNFENILTWDSGPEGTPDTVYSIEYKTYGERDWVAKKGCQRITRKSCNLTVETGNLTELYYARVTAVSAGGRSATKMTDRFSSLQHTTLKPPDVTCISKVRSIQMIVHPTPTPIRAGDGHRLTLEDIFHDLFYHLELQVNRTYQMHLGGKQREYEFFGLTPDTEFLGTIMICVPTWAKESAPYMCRVKTLPDRTW

SEQ ID NO: 7 - protein, 210 aa, Homo sapiens
TQSTHESLKPQRVQFQSRNFHNILQWQPGRALTGNSSVYFVQYKIYGQRQWKNKEDCWGTQELSCDLTSETSDIQEPYYGRVRAASAGSYSEWSMTPRFTPWWETKIDPPVMNITQVNGSLLVILHAPNLPYRYQKEKNVSIEDYYELLYRVFIINNSLEKEQKVYEGAHRAVEIEALTPHSSYCVVAEIYQPMLDRRSQRSEERCVEIP

SEQ ID NO: 8 - protein, 230 aa, Homo sapiens
VQFQSRNFHNILQWQPGRALTGNSSVYFVQYKIMFSCSMKSSHQKPSGCWQHISCNFPGCRTLAKYGQRQWKNKEDCWGTQELSCDLTSETSDIQEPYYGRVRAASAGSYSEWSMTPRFTPWWETKIDPPVMNITQVNGSLLVILHAPNLPYRYQKEKNVSIEDYYELLYRVFIINNSLEKEQKVYEGAHRAVEIEALTPHSSYCVVAEIYQPMLDRRSQRSEERCVEIP

SEQ ID NO: 9 - protein, 112 aa, Homo sapiens
VAGTQSTHESLKPQRVQFQSRNFHNILQWQPGRALTGNSSVYFVQYKIYGQRQWKNKEDCWGTQELSCDLTSETSDIQEPYYGRVRAASAGSYSEWSMTPRFTPWWERAKGL

SEQ ID NO: 10 - DNA, 666 bp, Homo sapiens
gttccctgtgtctctggtggtttgcctaaacctgcaaacatcaccttcttatccatcaacatgaagaatgtcctacaatggactccaccagagggtcttcaaggagttaaagttacttacactgtgcagtatttcatatatgggcaaaagaaatggctgaataaatcagaatgcagaaatatcaatagaacctactgtgatctttctgctgaaacttctgactacgaacaccagtattatgccaaagttaaggccatttggggaacaaagtgttccaaatgggctgaaagtggacggttctatccttttttagaaacacaaattggcccaccagaggtggcactgactacagatgagaagtccatttctgttgtcctgacagctccagagaagtggaagagaaatccagaagaccttcctgtttccatgcaacaaatatactccaatctgaagtataacgtgtctgtgttgaatactaaatcaaacagaacgtggtcccagtgtgtgaccaaccacacgctggtgctcacctggctggagccgaacactctttactgcgtacacgtggagtccttcgtcccagggccccctcgccgtgctcagccttctgagaagcagtgtgccaggactttgaaagatcaatcatcagagttcaaggctaaaatc

SEQ ID NO: 11 - DNA, 591 bp, Homo sapiens
gtggccattctgcctgcccctcagaacctctctgtactctcaaccaacatgaagcatctcttgatgtggagcccagtgatcgcgcctggagaaacagtgtactattctgtcgaataccagggggagtacgagagcctgtacacgagccacatctggatccccagcagctggtgctcactcactgaaggtcctgagtgtgatgtcactgatgacatcacggccactgtgccatacaaccttcgtgtcagggccacattgggctcacagacctcagcctggagcatcctgaagcatccctttaatagaaactcaaccatccttacccgacctgggatggagatcaccaaagatggcttccacctggttattgagctggaggacctggggccccagtttgagttccttgtggcctactggaggagggagcctggtgccgaggaacatgtcaaaatggtgaggagtgggggtattccagtgcacctagaaaccatggagccaggggctgcatactgtgtgaaggcccagacattcgtgaaggccattgggaggtacagcgccttcagccagacagaatgtgtggaggtgcaagga

SEQ ID NO: 12 - DNA, 345 bp, Homo sapiens
gcacaaattggcccaccagaggtggcactgactacagatgagaagtccatttctgttgtcctgacagctccagagaagtggaagagaaatccagaagaccttcctgtttccatgcaacaaatatactccaatctgaagtataacgtgtctgtgttgaatactaaatcaaacagaacgtggtcccagtgtgtgaccaaccacacgctggtgctcacctggctggagccgaacactctttactgcgtacacgtggagtccttcgtcccagggccccctcgccgtgctcagccttctgagaagcagtgtgccaggactttgaaagatcaatcatcagagttcaaggct

SEQ ID NO: 13 - DNA, 141 bp, Homo sapiens
gttccctgtgtctctggtggtttgcctaaacctgcaaacatcaccttcttatccatcaacatgaagaatgtcctacaatggactccaccagagggtcttcaaggagttaaagttacttacactgtgcagtatttcatctcc

SEQ ID NO: 14 - DNA, 141 bp, Homo sapiens
gttccctgtgtctctggtggtttgcctaaacctgcaaacatcaccttcttatccatcaacatgaagaatgtcctacaatggactccaccagagggtcttcaaggagttaaagttacttacactgtgcagtatttcatcaca

SEQ ID NO: 15 - DNA, 630 bp, Homo sapiens
cctgaggacccctcggatctgctccagcacgtgaaattccagtccagcaactttgaaaacatcctgacgtgggacagcgggccagagggcaccccagacacggtctacagcatcgagtataagacgtacggagagagggactgggtggcaaagaagggctgtcagcggatcacccggaagtcctgcaacctgacggtggagacgggcaacctcacggagctctactatgccagggtcaccgctgtcagtgcgggaggccggtcagccaccaagatgactgacaggttcagctctctgcagcacactaccctcaagccacctgatgtgacctgtatctccaaagtgagatcgattcagatgattgttcatcctacccccacgccaatccgtgcaggcgatggccaccggctaaccctggaagacatcttccatgacctgttctaccacttagagctccaggtcaaccgcacctaccaaatgcaccttggagggaagcagagagaatatgagttcttcggcctgacccctgacacagagttccttggcaccatcatgatttgcgttcccacctgggccaaggagagtgccccctacatgtgccgagtgaagacactgccagaccggacatgg

SEQ ID NO: 16 - DNA, 633 bp, Homo sapiens
actcagtcaacgcatgagtctctgaagcctcagagggtacaatttcagtcccgaaattttcacaacattttgcaatggcagcctgggagggcacttactggcaacagcagtgtctattttgtgcagtacaaaatatatggacagagacaatggaaaaataaagaagactgttggggtactcaagaactctcttgtgaccttaccagtgaaacctcagacatacaggaaccttattacgggagggtgagggcggcctcggctgggagctactcagaatggagcatgacgccgcggttcactccctggtgggaaacaaaaatagatcctccagtcatgaatataacccaagtcaatggctctttgttggtaattctccatgctccaaatttaccatatagataccaaaaggaaaaaaatgtatctatagaagattactatgaactactataccgagtttttataattaacaattcactagaaaaggagcaaaaggtttatgaaggggctcacagagcggttgaaattgaagctctaacaccacactccagctactgtgtagtggctgaaatatatcagcccatgttagacagaagaagtcagagaagtgaagagagatgtgtggaaattccatga

SEQ ID NO: 17 - DNA, 37 bp, Synthetic
ccgccgggcgcgccaccatgcagactttcacaatggt

SEQ ID NO: 18 - DNA, 32 bp, Synthetic
ccgccggatatctccttgcacctccacacatt

SEQ ID NO: 19 - DNA, 36 bp, Synthetic
ccgccggcgcgccaccatgcgggctcccggccgccc

SEQ ID NO: 20 - DNA, 32 bp, Synthetic
ccgccgatatcttttagccttgaactctgatg

SEQ ID NO: 21 - DNA, 18 bp, Synthetic
agctgccttcttcacttg

SEQ ID NO: 22 - DNA, 18 bp, Synthetic
ttgctctgcctcttattc

(19) United States
(12) Patent Application Publication
(10) Pub. No.: US 2004/0204351 A1
(43) Pub. Date: Oct. 14, 2004

(54) Soluble proteins that inhibit cytokine signal transduction pathways

(76) Inventors: Scott William Rowlinson, Indianapolis, IN (US); David Bruce Baldwin, Carmel, IN (US)

Correspondence Address: MaryAnn Wiskerchen, Eli Lilly & Company, Patent Division, PO Box 6288, Indianapolis, IN, 46206-6288, US

(21) Appl. No.: 10/490,593

(22) PCT Filed: Oct. 9, 2002
(86) PCT No.: PCT/US02/29844

(30) Foreign Application Priority Data
Oct. 22, 2001 (US) 60342233

(51) Int. Cl. A61K 38/17

(52) U.S. Cl. 514/12

(57) ABSTRACT

This invention provides methods using secreted IL10-related cytokine receptor subunits, and variants and fusions and combinations thereof for treating and preventing, inflammatory system, immune system, and cardiovascular system disorders as well as hematopoiesis disorders and cancer.

### FIELD OF THE INVENTION

The present invention relates to therapeutic uses of isolated, soluble polypeptides, designated herein as IL20Rβs, IL20Rαs, LP338s, LP339s, LP340s, IL22Rs, IL22RA2s, IL22BP1s, IL22BP2s.

### BACKGROUND OF THE INVENTION

Cytokines are soluble proteins that mediate reactions between cells and influence cell growth and differentiation. Cytokines exert their effects by binding to specific cell-surface receptors that leads to activation of cytokine-specific signal transduction pathways. Certain cytokines, interleukin-20 (IL20), interleukin-22 (IL22), interleukin-19 (IL19), and interleukin-24 (IL24, also referred to as mda-7), have limited homology (less than 30%) to interleukin-10 (IL10) and are often referred to as IL10-related cytokines. IL10 is a cytokine with immunosuppressive and anti-inflammatory properties. IL10 is a regulator of numerous myeloid and lymphoid cell activities and indirectly inhibits the production of various inflammatory cytokines by both T-cells and NK cells (for review see ref. 1).

IL20 is a recently discovered IL10-related cytokine formerly called Zcyto 10 (International Patent Publication WO 99/27103). The IL20 coding sequence maps to human chromosome 1q32. This is the same region to which the genes encoding IL10, IL19 (44) and IL24 map (2). IL20 activates STAT3-containing signal transduction pathways via STAT3-responsive reporters. IL20 has also been shown to up-regulate IL-8, an inducer of angiogenesis and a chemoattractant for neutrophils (International patent publication WO 01/46261).

IL22 activates the JAK-STAT signaling pathway and modestly inhibits IL-14 production (3). Also, there are elevated IL22 mRNA levels in stimulated T-cells. The biological activities of IL24 and IL19 are not well understood although expression of IL19 is induced in activated monocytes (44) and IL24 reportedly induces apoptosis in certain tumor cells and cell lines by an unknown mechanism (3).

Cytokine receptors are composed of one or more integral membrane proteins that bind a cytokine with high affinity and transduce the binding event into the cell through the cytoplasmic portions of the receptor subunits. Class II cytokine receptors, such as those that bind IL10-related cytokines, are typically heterodimers composed of two distinct receptor chains, the α and β chains. In general, the α subunits are the primary cytokine-binding proteins, and the β subunits are required for formation of high affinity binding sites and for signal transduction. However, Blumberg, et al. (2) claim that for the IL20 receptor, both subunits are required for IL20 binding.

The class II cytokine receptor subunits are not always exclusive to the binding of a single type of cytokine. For example, the IL10 receptor complex is composed of IL10Rα, the ligand-binding subunit, and IL10Rβ, the signaling subunit (1), while IL22 also uses IL10Rβ in combination with IL22R (4) in its receptor complex. Each receptor subunit, IL10Rβ and IL22R, alone is able to bind IL22, but both subunits are required for signal transduction. IL20, IL24 and IL19 all have been shown to bind the receptor complex composed of IL20Rα and IL20Rβ (5). Both IL24 and IL20 also bind to a receptor complex composed of IL22R and IL20Rβ, (5). These relationships are diagrammed in FIG. 18 herein.

International Patent Publication WO 01/4623 and U.S. Pat. No. 5,945,511 teach the nucleic acid sequence and the protein sequence of the a subunit of the IL20 receptor, formerly called Zcytor7. U.S. patent application Ser. No. 60/295088 teaches the nucleic acid sequence and the protein sequence of three different IL20 receptor α (IL20Rα) subunit splice variants, LP338, LP339 and LP340, diagrammed in FIG. 17 herein. International Patent Publication WO 99/46379 teaches the nucleic acid sequence and the protein sequence of the β subunit of the IL20 receptor (IL20Rβ), formerly called DIRS1.

International Patent Publications WO 01/46232 and WO 01/46261 and U.S. Pat. No. 5,945,511 further teach the use of soluble IL20 receptors, (i.e., the extracellular portion) for down-regulating the effects of IL20 binding to its natural receptor. However, these references are contradictory in that U.S. Pat. No. 5,945,511 states that the extracellular IL20Rα subunit, in the absence of the IL20Rβ subunit, can mediate IL20 down-regulation, while subsequent references WO 01/46232 and WO 01/46261 use only linked extracellular IL20Rα and extracellular IL20Rβ to facilitate IL20 down-regulation and teach that IL20 cannot bind to only one of the subunits in the absence of the other. International Patent Publication WO 99/46379 teaches the existence of an IL20Rβ subunit and hypothesizes that its soluble extracellular domain will bind to its corresponding biological ligand with high affinity but offers no evidence that this occurs.

The polynucleotide sequence and amino acid sequence of IL22 receptor subunit, IL22R (also called Zcytor11), are taught in U.S. Pat. No. 5,965,704. There is also a naturally occurring soluble IL22 receptor subunit named IL22RA2 with Genbank accession number AYO44429 (42) and two splice variants of IL22RA2 named IL22BP1 and IL22BP2 with Genbank accession numbers AY040567 and AY040568 respectively. The polynucleotide sequence and amino acid sequence of IL22 is taught in International Patent Publication WO 99/61617.

There is great clinical need for effective methods to treat or prevent diseases that involve IL10-related cytokines. Numerous such diseases and disorders exist including, but not limited to, inflammation, immune system cardiovascular and hematopoietic disorders and regulation of cellular proliferation. One such method would be to administer an agent that would effectively inhibit the binding of a cytokine to its normal receptor and thereby inhibit the subsequent signal transduction pathway.

### SUMMARY OF THE INVENTION

The present invention addresses the need for molecules useful in the treatment and/or prevention of inflammation, immune system, cardiovascular and hematopoietic disorders and regulation of cellular proliferation by inhibiting the binding of a cytokine (e.g., IL19, IL20 and/or IL24) to its receptor complex, thereby preventing signal transduction.

One embodiment of the present invention is a method for using purified and isolated, soluble receptor proteins IL20Rαs (SEQ ID NO. 1), IL20Rβs (SEQ ID NO. 2), LP338s (SEQ ID NO. 3), LP339s (SEQ ID NO. 4), LP340s (SEQ ID NO. 5), IL22Rs (SEQ ID NO. 6) and IL22RA2s (SEQ ID NO. 7) and IL22RA2 soluble splice variants named IL22BP1s (SEQ ID NO. 8) and IL22BP2s (SEQ ID NO. 9) and/or variants, complexes and combinations thereof to down-regulate IL19, IL20 and/or IL24 activity or combinations thereof in vivo, in situ, and in vitro. The nomenclature of the soluble receptor proteins of the invention (i.e., the extracellular domain of the full-length receptor protein) and the polynucleotides encoding them is summarized in Table 2 herein.

Another embodiment is a method for using fusion proteins comprising a purified and isolated, soluble receptor protein selected from the group consisting of IL20Rαs (SEQ ID NO. 1), IL20Rβs (SEQ ID NO. 2), LP338s (SEQ ID NO. 3), LP339s (SEQ ID NO. 4), LP340s (SEQ ID NO. 5), IL22Rs (SEQ ID NO. 6), IL22RA2s (SEQ ID NO. 7), IL22BP1s (SEQ ID NO. 8), IL22BP2s (SEQ ID NO. 9) and variants, complexes and combinations thereof to down-regulate IL19, IL20 and/or IL24 activity or combinations thereof in vivo, in situ, and/or in vitro.

The present invention contemplates compositions comprising purified and isolated, soluble receptors comprised of a soluble IL20Rβ subunit and a soluble IL20Rα splice-variant subunit, wherein the soluble IL20Rβ subunit is comprised of a polypeptide having an amino acid sequence selected from the group consisting of SEQ ID No. 2, variants thereof and fusion proteins comprising SEQ ID No. 2 and variants thereof, and the IL20Rα splice-variant subunit is comprised of a polypeptide having an amino acid sequence selected from the group consisting of SEQ ID NOs: 3, 4, 5, variants thereof and fusion proteins comprising SEQ ID NOs: 3, 4, 5, or variants thereof and complexes of such fusion proteins wherein the fusion proteins are linked.

The soluble IL20Rβ subunit and the soluble IL20Rα splice variant subunit may exist as a mixture of independent subunits (“combination”) or they are optionally linked together through the soluble receptor proteins or through heterologous protein fusion partners or both. The linking between soluble IL20Rβ subunit and soluble IL20Rα splice variant subunit can be by any means, but preferably by a peptide bond or a disulfide bond between a polypeptide fusion partner covalently linked to the soluble IL20Rβ subunit and a polypeptide fusion partner covalently linked to the soluble IL20Rα splice variant subunit. The linking can also be by means of a polypeptide linker. The invention embodies the method of using these soluble receptors for downregulating IL20, IL19 and/or IL24 activity or any combination thereof in vivo, in situ, and/or in vitro.

The present invention further embodies compositions comprising purified and isolated, soluble receptors comprised of a soluble IL20Rβ subunit and a soluble IL20Rα subunit, wherein the soluble IL20Rβ subunit is comprised of a polypeptide having an amino acid sequence selected from the group consisting of SEQ ID No. 2, variants thereof and fusion proteins comprising SEQ ID No:2 and the soluble IL20Rα subunit is comprised of a polypeptide having an amino acid sequence selected from the group consisting of SEQ ID No: 1, variants thereof and fusion proteins comprising SEQ ID No:1.

The soluble IL20Rβ subunit and the soluble IL20Rα subunit may exist as a mixture of independent subunits (a “combination”) or they are preferably linked together by a polypeptide linker. The linking between a soluble IL20Rβ subunit and a soluble IL20Rα subunit can be by any means, but preferably by a peptide bond or a disulfide bond between a polypeptide fusion partner covalently linked to the soluble IL20Rβ subunit and a polypeptide fusion partner covalently linked to the IL20Rα subunit. The invention embodies methods of using these soluble receptors for downregulating IL20, IL19 and/or IL24 activity or any combination thereof in vivo, in situ, and/or in vitro.

The present invention embodies compositions comprising purified and isolated, soluble receptors comprised of a soluble IL20Rβ subunit and a subunit selected from the group consisting of a soluble IL22R subunit, a soluble IL22RA2 subunit, a soluble IL22BP1 subunit and a soluble IL22BP2 subunit, wherein the soluble IL20Rβ subunit is comprised of a polypeptide having an amino acid sequenced selected from the group consisting of SEQ ID No. 2, variants thereof, and fusion proteins comprising SEQ ID NO. 2 and the soluble IL22R subunit is comprised of a polypeptide selected from the group consisting of SEQ ID No. 6, variants therof and fusion proteins comprising SEQ ID No. 6 and the soluble IL22RA2 subunit is comprised of a polypeptide selected from the group consisting of SEQ ID No. 7, variants thereof and fusion proteins comprising SEQ ID No. 7 and the soluble IL22BP1 subunit is comprised of polypeptide selected from the group consisting of SEQ ID No. 8, variants thereof and fusion proteins comprising SEQ ID No. 8 and the soluble IL22BP2 subunit is comprised of polypeptide selected from the group consisting of SEQ ID NO. 9, variants thereof and fusion proteins comprising SEQ ID No. 9.

The IL20Rβ subunit and the IL22R or IL22RA2 or IL22BP1 or IL22BP2 subunit may exist as a mixture of independent subunits (a “combination”) or they are preferably linked together by a polypeptide linker. The linking between IL20Rβ subunit and IL22R or IL22RA2 or IL22BP1 or IL22BP2 subunit can be by any means, but preferably by a peptide bond or a disulfide bond between a polypeptide connected to the IL20Rβ subunit and a polypeptide connected to the IL22R or IL22RA2 or IL22BP1 or IL22BP2 subunit. The invention embodies the method of using these soluble receptors for downregulating IL20 and/or IL24 activity in vivo, in situ, and/or in vitro.

The invention further embodies compositions comprising an isolated and purified fusion polypeptide consisting essentially of a first portion and a second portion joined by a third portion. The first portion of the fusion polypeptide comprises (a) a protein with a sequence shown in SEQ ID Nos. 1, 2, 3, 4, 5, 6, 7, 8 or 9 (b) a polypeptide that has an amino acid sequence at least 90%, preferably at least 91%, 92%, 93% or 94%, more preferably at least 95%, even more preferably at least 96%, 97%, or 98% and most preferably at least 99% identical (i.e., amino acid sequence identity) to that described in (a). The second portion of the fusion polypeptide consists of another polypeptide. Within one embodiment the fusion partner (i.e., the second portion) is a constant region of the heavy chain of an immunoglobulin (Ig) molecule or a portion thereof or the constant region of the light chain of an Ig molecule. Such constant region sequences are readily available through databases including Genbank (e.g., Genbank accession numbers X68518, X57331, X67292, X67301, L43100). Within another embodiment the fusion partner (i.e., the second portion) is an affinity tag (e.g. FLAG (Asp-Tyr-Lys-Asp-Asp-Asp-Asp-Lys) or 6×His or an immunoglobulin Fc polypeptide) or a reporter molecule (e.g. luciferase or beta-galactosidase). Within yet another emodiment, the fusion partner (i.e., the second portion) comprises (a) a protein with a sequence shown in SEQ ID Nos. 1, 2, 3, 4, 5, 6, 7, 8 or 9, or (b) a protein that has an amino acid sequence at least 90%, preferably at least 91%, 92%, 93% or 94%, more preferably at least 95%, even more preferably at least 96%, 97%, or 98% and most preferably at least 99% identical (i.e., amino acid sequence identity) to that described in (a). (These embodiments exemplify possible second portions of the fusion protein and are not intended to be limitations). The third portion, separating the first portion and the second portion of the fusion protein, is selected from the group consisting of a peptide bond or a polypeptide linker sequence. The invention further contemplates more than one of these fusion polypeptides being linked together to form a “complex” of fusion polypeptides. These fusion polypeptides and complexes of fusion polypeptides are useful for the method of downregulating IL20, IL19 and/or IL24 activity or any combination thereof in vivo, in situ, and/or in vitro.

In other embodiments, the invention provides a novel method of modulating the physiology or development of a cell in vivo or in situ comprising introducing into such cell, or the environment of such cell, a therapeutically effective amount of a composition comprising a protein with sequence shown in SEQ ID NO. 1, 2, 3, 4, 5, 6, 7, 8 or 9, or a variant thereof, or combinations or complexes thereof or a fusion protein comprising a protein with a sequence shown in SEQ ID NO. 1, 2, 3, 4, 5, 6, 7, 8 or 9, or a variant thereof or combinations or complexes thereof for the purpose of treating or preventing diseases that involve IL10-related cytokine(s) by inhibiting binding of a cytokine to its normal receptor and thereby inhibiting the subsequent signal transduction pathway. Numerous such diseases and disorders exist including, but not limited to, inflammation, immune system, cardiovascular and hematopoietic disorders and regulation of cellular proliferation.

### BRIEF DESCRIPTION OF THE FIGURES.

FIG. 1 represents the amino acid sequence of soluble IL20 Receptor Subunit α (IL20Rαs, i.e., the extracellular domain) (SEQ ID No. 1).

FIG. 2 represents the amino acid sequence of soluble IL20 Receptor Subunit β (IL20Rβs, SEQ ID No. 2).

FIG. 3 represents the amino acid sequence of soluble IL20 Receptor Subunit α, splice variant (LP338s, SEQ ID No. 3).

FIG. 4 represents the amino acid sequence of soluble IL20 Receptor Subunit α, splice variant (LP339s, SEQ ID No. 4).

FIG. 5 represents the amino acid sequence of soluble IL20 Receptor Subunit α, splice variant (LP340s, SEQ ID No. 5).

FIG. 6 represents the amino acid sequence of soluble IL22 Receptor (IL22Rs, SEQ ID No. 6).

FIG. 7 represents the amino acid sequence of soluble IL22RA2 (SEQ ID NO. 7).

FIG. 8 represents the amino acid sequence of the soluble form of IL22BP1 (Genbank Accession number AY040567) without the signal peptide sequence (SEQ ID NO. 8).

FIG. 9 represents the amino acid sequence of the soluble form of IL22BP2 (Genbank Accession number AY040568) without the signal peptide sequence (SEQ ID NO. 9).

FIG. 10 represents the polynucleotide sequence encoding SEQ ID NO. 1 (SEQ ID No. 10).

FIG. 11 represents the polynucleotide sequence encoding SEQ ID NO. 2 (SEQ ID No. 11).

FIG. 12 represents the polynucleotide sequence encoding SEQ ID NO. 3 (SEQ ID No. 12).

FIG. 13 represents the polynucleotide sequence encoding SEQ ID NO. 4 (SEQ ID No. 13).

FIG. 14 represents the polynucleotide sequence encoding SEQ ID NO. 5 (SEQ ID No. 14).

FIG. 15 represents the polynucleotide sequence encoding SEQ ID NO. 6 (SEQ ID No. 15).

FIG. 16 represents the polynucleotide sequence encoding SEQ ID NO. 7 (SEQ ID NO. 16)

FIG. 17 is a schematic of IL20 receptor alpha (IL20Rα) subunit receptor and splice variants of IL20Rα subunit.

FIG. 18 is a schematic of receptor complexes capable of binding IL19, IL20, and IL24.

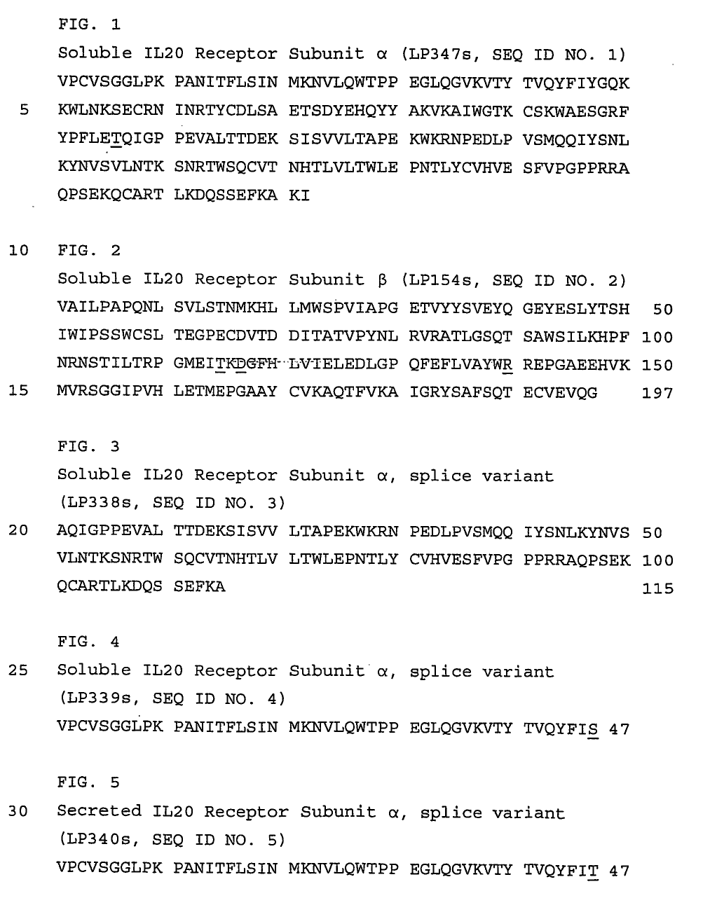

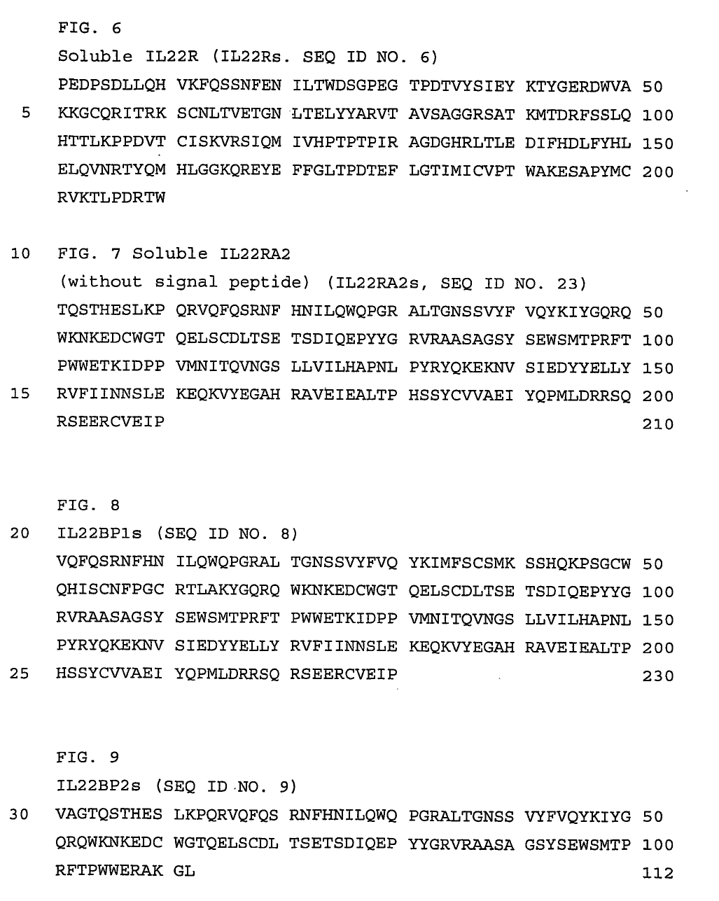

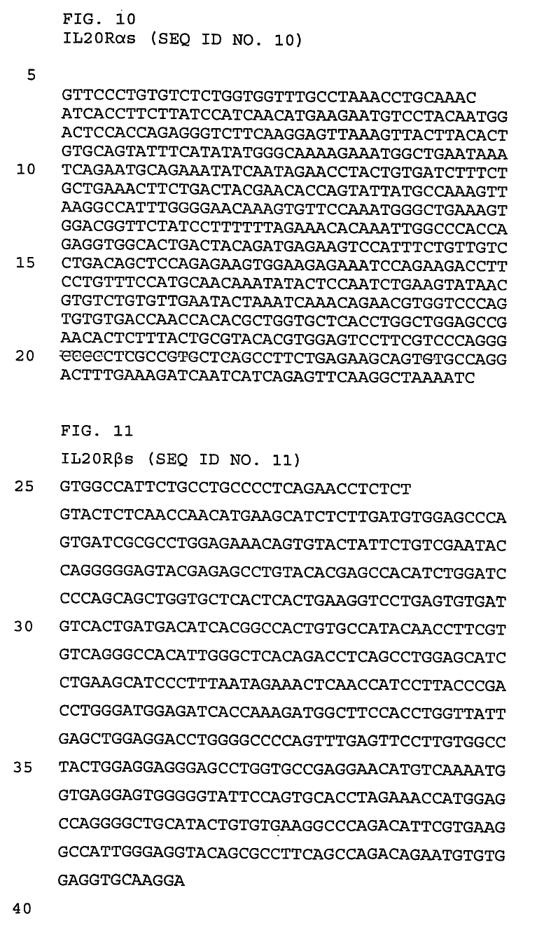

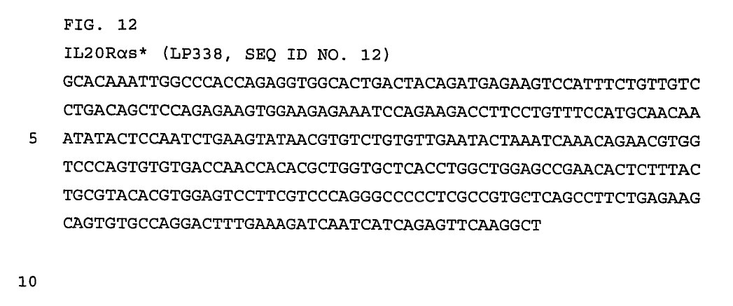

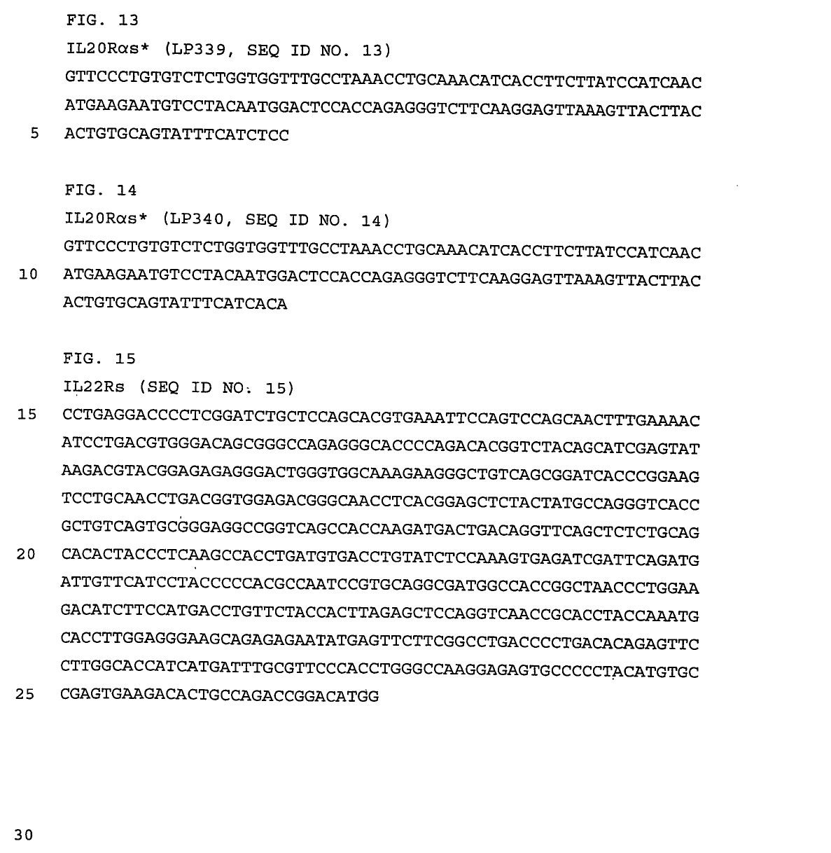

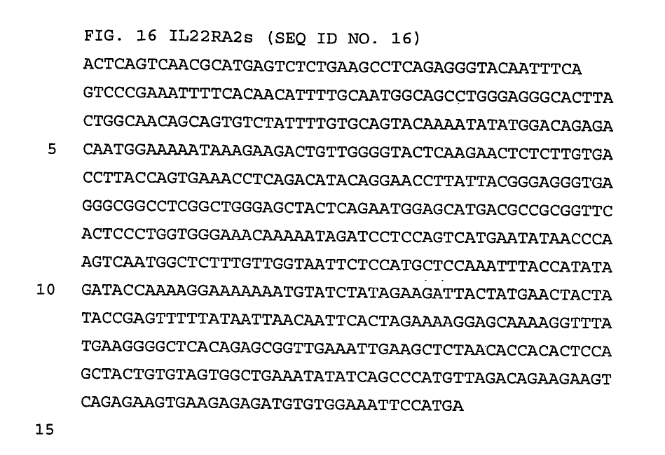

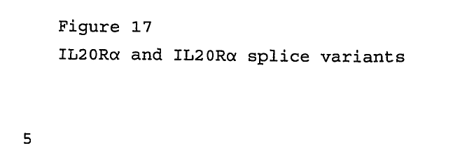

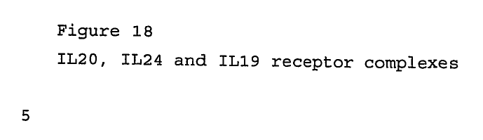

### DETAILED DESCRIPTION OF THE INVENTION

The invention is not limited to the particular embodiments described below, as variations may be made and still fall within the scope of the appended claims. The terminology used herein is for the purpose of describing particular embodiments, and is not intended to be limiting. Instead, the scope of the present invention will be established by the appended claims.

Definitions

The terms “LP polypeptide(s)” and “LP” as used herein refer to various polypeptides. The complete designation of LP immediately followed by a number (e.g., LP154) refers to a particular polypeptide sequence as described herein. The polypeptides of the invention described herein may be isolated from a variety of sources including, but not limited to, tissue culture media of mammalian cells expressing the polypeptide, lysed E.coli expressing the polypeptide, yeast, or Sf9 cells expressing the polypeptide, or the polypeptide prepared by recombinant or synthetic methods.

The term “isolated” when used in relation to a nucleic acid or protein, means the material is identified and separated from at least one contaminant with which it is ordinarily associated in its natural source. Such a nucleic acid could be part of a vector and/or such nucleic acid or protein could be part of a composition, and still be isolated in that such vector or composition is not part of its natural environment.

As used herein, the term “purified” means the result of any process that removes from a sample a contaminant from the component of interest, such as a protein or nucleic acid. The percent of a purified component is thereby increased in the sample.

As used herein, the terms “complementary” or “complementarity” are used in reference to nucleic acids (i.e., a sequence of nucleotides) related by the well-known base-pairing rules that A pairs with T and C pairs with G. For example, the sequence 5′-A-G-T-3′, is complementary to the sequence 3′-T-C-A-5′.

The term “receptor” as used herein denotes a normally, cell-associated protein that binds to a bioactive molecule (i.e., a ligand) and mediates the effect of the ligand on the cell. Receptors may be modified to be a “soluble receptor” by removal of sequence that joins it to the cell-membrane. Membrane-bound receptors are characterized by a multi-domain structure comprising an extracellular ligand-binding domain and an intracellular effector domain that is typically involved in signal transduction. Binding of ligand to receptor results in a change in the receptor that causes an interaction between the effector domain and other molecule(s) in the cell. This interaction in turn leads to an alteration in the metabolism of the cell. Metabolic events that are linked to receptor-ligand interactions include, but are not limited to, gene transcription, phosphorylation, dephosphorylation, increase in cAMP production, mobilization of cellular calcium, mobilization of membrane lipids, cell adhesion, hydrolysis of inositol lipids and hydrolysis of phospholipis. In general, receptors can be membrane bound, cytosolic or nuclear, monomeric or multimeric.

A “soluble receptor” as used herein refers to the extracellular domain of a receptor, separated from enough of the signal peptide and the transmembrane domain such that it no longer is membrane-bound.

In the present invention “extracellular” refers to a form of the polypeptide that is essentially free of the signal peptide and the transmembrane and cytoplasmic domains of the full-length polypeptide. The exact boundaries of where the signal peptide ends and the extracellular domain begins and the exact boundaries of where the extracellular domain ends and the transmembrane domain begins may vary but most likely by no more than about six amino acids at either end of the domain as identified herein. Therefore, an extracellular signal peptide/extracellular domain boundary as identified in the examples, figures, or specification may be shifted in either direction (upstream or downstream) by 6, 5, 4, 3, 2, 1, or 0 amino acids. Additionally, an extracellular domain/transmembrane domain boundary as identified in the examples, figures, or specification may be shifted in either direction by 6, 5, 4, 3, 2, 1 or 0 amino acids. All such polypeptides and the nucleic acid molecules encoding them are contemplated by the present invention.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

“Conservative amino acid substitutions” are those substitutions that, when made, least interfere with the properties of the original protein, i.e., the structure and especially the function of the protein is conserved and not significantly changed by such substitutions. Table 1 below shows preferred conservative amino acid substitutions for an original amino acid in a protein with the most preferred substitution in bold type.

TABLE 1

| Original Residue | Conservative Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| Gly (G) | Ala, Pro |
| His (H) | Arg, Asn, Gln, Lys |
| Ile (I) | Leu, Val, Met, Ala, Phe, norleucine |
| Leu (L) | Ile, norleucine, Val, Met, Ala, Phe |
| Lys (K) | Arg, Gln, Asn |
| Met (M) | Leu, Ile, Phe |
| Phe (F) | Leu, Val, Ile, Ala, Try |
| Pro (P) | Ala |
| Ser (S) | Thr |
| Thr (T) | Ser |
| Trp (W) | Tyr, Phe |
| Tyr (Y) | Phe, Trp, Thr, Ser |
| Val (V) | Leu, Ile, norleucine, Ala, Phe |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

It is also well known that different species exhibit prefered codon usage (6, 7, 8), that is, translation codons that are most frequently used in cells of a certain species, thus favoring one or a few representatives of the possible codons encoding each amino acid. Such preferential codons for a particular species can be introduced into the polynucleotides of the present invention by various methods known well in the art. These codons may enhance production of the protein by making protein translation more efficient within a particular cell type or species.

It is recognized that according to the present invention, when a polynucleotide is claimed as described herein, it is understood that what is claimed are the sense strand, the anti-sense strand and the double-stranded DNA. Also claimed is the messenger RNA (mRNA) that encodes the polypeptides of the present invention.

The term “mature protein” or “mature polypeptide” as used herein refers to the form(s) of the protein as would be produced by expression in a mammalian cell. For example, it is generally hypothesized that once export of a growing protein chain across the rough endoplasmic reticulum has been initiated, proteins secreted by mammalian cells have a signal peptide (SP) sequence which is cleaved from the complete polypeptide to produce a “mature” form of the protein. Oftentimes, cleavage of a secreted protein is not uniform and may result in more than one species of mature protein. The cleavage site of a secreted protein is determined by the primary amino acid sequence of the complete protein and generally cannot be predicted with complete accuracy. Methods for predicting whether a protein has an SP sequence, as well as the cleavage point for that sequence, are known in the art. A cleavage point may exist within the N-terminal domain between amino acid 10 and amino acid 35. More specifically the cleavage point is likely to exist after amino acid 15 but before amino acid 31. As one of ordinary skill would appreciate, however, cleavage sites sometimes vary from organism to organism and may even vary from molecule to molecule within a cell and cannot be predicted with absolute certainty. Optimally, cleavage sites for a secreted protein are determined experimentally by amino-terminal sequencing of the one or more species of mature proteins found within a purified preparation of the protein.

Nucleic acid is “operably linked” when it is placed into a functional relationship with another nucleic acid sequence. Generally, “operably linked” means that the DNA sequences being linked are contiguous, and, in the case of a secretory leader, contiguous and in reading phase. However, enhancers do not have to be contiguous.

The term “amino acid” is used herein in its broadest sense, and includes naturally occurring amino acids as well as non-naturally occurring amino acids, including amino acid analogs and derivatives. The latter includes molecules containing an amino acid moiety. One skilled in the art will recognize, in view of this broad definition, that reference herein to an amino acid includes naturally occurring proteogenic L-amino acids; D-amino acids; chemically modified amino acids, such as amino acid analogs and derivatives; naturally occurring non-proteogenic amino acids such as norleucine, β-alanine, ornithine, etc.; and chemically synthesized compounds having properties known in the art to be characteristic of amino acids. As used herein, the term “proteogenic” indicates that the amino acid can be incorporated into a peptide, polypeptide, or protein in a cell through a metabolic pathway.

The terms “treating”, “treatment” and “therapy” as used herein refer to curative therapy, prophylactic therapy, and preventive therapy. An example of “preventive therapy” is the prevention or lessened targeted pathological condition or disorder. Those in need of treatment include those already with the disorder as well as those prone to have the disorder or those in whom the disorder is to be prevented.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption but, rather, is cyclic in nature.

Administration “in combination with” one or more further therapeutic agents includes simultaneous (concurrent) and consecutive administration in any order.

A “therapeutically-effective amount” is the minimal amount of active agent (e.g., an LP polypeptide) necessary to impart therapeutic benefit to a mammal, i.e., an amount that induces, ameliorates or otherwise causes an improvement in the pathological symptoms, disease progression, physiological conditions associated with or resistance to succumbing to the aforedescribed disorder.

“Active” or “activity” in the context of variants of the polypeptides of the invention refers to retention of a biologic function of the polypeptide of the invention and/or the ability to bind to a receptor or ligand much as would an unmodified polypeptide of the invention. More specifically, “biological activity” refers to a biological function (either inhibitory or stimulatory) caused by a reference polypeptide. Exemplary biological activities include, but are not limited to, the ability of such molecules to induce or inhibit infiltration of inflammatory cells (e.g., leukocytes) into a tissue, to induce or inhibit adherence of a leukocyte to an endothelial or epithelial cell, to stimulate or inhibit T-cell proliferation or activation, to stimulate or inhibit cytokine release by cells or to increase or decrease vascular permeability.

Soluble Receptor Subunits that Inhibit Cytokine Activity

The present invention embodies a method of using purified and isolated, soluble receptor proteins IL20Rβs (SEQ ID NO. 2), IL20Rαs (SEQ ID NO. 1), LP338s (SEQ ID NO. 3), LP339s (SEQ ID NO. 4), LP340s (SEQ ID NO. 5), IL22Rs (SEQ ID NO. 6) and IL22RA2s (SEQ ID NO. 7) and IL22RA2 soluble splice variants named IL22BP1s (SEQ ID NO. 8) and IL22BP2s (SEQ ID NO. 9) and variants, complexes and combinations thereof to down-regulate IL19, IL20 and/or IL24 activity or combinations thereof.

IL20 is defined and methods for producing it are contained in International Patent Application No. PCT/US98/25228, publication WO 99/27103 and U.S. patent application Ser. No. 09/313,458 filed May 17,1999.

The IL20 receptor has been discovered and is a heterodimer comprised of the polypeptide termed ‘IL20Rα’ (formerly called Zcytor7) and a polypeptide termed ‘IL20Rβ’ (formerly called DIRS1). The IL20Rα polypeptide, nucleic acid that encodes it, and methods for producing it are disclosed in U.S. Pat. No. 5,945,511. The extracellular domain of the human IL20Rα polypeptide is identified in International Patent Publication WO 01/46232 and its sequence is represented herein by SEQ ID No. 1 (polypeptide) and SEQ ID NO. 10 (polynucleotide) and referred to herein as IL20Rαs. U.S. patent application Ser. No. 60/295088 identifies the polypeptide sequence and the nucleic acid encoding three splice-variant forms of IL20Rα. The sequence of the soluble form of these IL20Rα splice-variants is shown in SEQ ID NOs. 3-5 (polypeptide) and 12-14 (polynucleotide). The soluble IL20Rβ polypeptide has the sequence shown in SEQ ID NO. 2 and the nucleic acid that encodes it has the sequence shown in SEQ ID NO. 11. The IL20Rα/IL20Rβ receptor complex is capable of binding IL19 and IL24 while IL20Rβ/IL22R is capable of binding IL20 and IL24 (5).

One subunit of the IL22 receptor is termed IL22R. The IL22R polypeptide, nucleic acid that encodes it, and methods for producing it are disclosed in U.S. Pat. No. 5,965,704 and its soluble form is represented herein by SEQ ID NO. 6(polypeptide) and 15 (polynucleotide. The cloning of IL22RA2, a soluble IL22 antagonist, is described in reference 42.

Table 2a hereinbelow summarizes the receptor subunits, complexes, and fusions as taught in the present invention that may have cytokine antagonist or inhibitor activity through modification of the active signalling complex, such that subsequent signal transduction may be prevented or altered. Table 2b hereinbelow summarizes the inhibitor nomenclature.

TABLE 2a

| Inhibitor* | Cytokine Inhibited | | |
|---|---|---|---|
| IL20Rβs | IL20 | IL19 | IL24 |
| IL20Rαs | | IL19 | IL24 |
| LP338s | | IL19 | IL24 |
| LP339s | | IL19 | IL24 |
| LP340s | | IL19 | IL24 |
| IL22Rs | IL20 | | IL24 |
| IL20Rβs/IL20Rαs* | IL20 | IL19 | IL24 |
| IL20Rβs/LP338s* | IL20 | IL19 | IL24 |
| IL20Rβs/LP339s* | IL20 | IL19 | IL24 |
| IL20Rβs/LP340s* | IL20 | IL19 | IL24 |
| IL20Eβs/IL22Rs* | IL20 | | IL24 |
| IL20Rβs/IL22RA2s* | IL20 | | IL24 |
| IL20Rβs/IL22BP1s* | IL20 | | IL24 |
| IL20Rβs/IL22BP2s* | IL20 | | IL24 |

*two soluble receptor subunits linked, fused or combined

TABLE 2b

| Protein Description | | SEQ ID NO. |
|---|---|---|
| IL20Rαs | soluble IL20R α-subunit | 1 |
| IL20Rβs | soluble IL20R β-subunit | 2 |
| LP338s | soluble IL20R α-subunit | 3 |
| | splice variant | |
| LP339s | soluble IL20R α-subunit | 4 |
| | splice variant | |
| LP340s | soluble IL20R α-subunit | 5 |
| | splice variant | |
| IL22Rs | soluble IL22R subunit | 6 |
| IL22RA2s | soluble natural IL22 agonist | 7 |
| IL22BP1s | soluble splice variant of IL22RA2 | 8 |
| IL22BP2s | soluble splice variant of IL22RA2 | 9 |

The polypeptides of the present invention are the soluble, extracellular domains of cytokine receptors IL20Rα, IL20Rα splice-variants, IL20Rβ, IL22R, IL22RA2, IL22BP1, IL22BP2 and variants, combinations, complexes and fusion proteins thereof. These molecules lack the transmembrane domain and cytoplasmic domain that are present in the full-length, membrane-bound cytokine receptor molecules. The polypeptides of the invention may have a signal peptide sequence to enable protein transport within the cell; however, this signal peptide sequence is not present in the mature polypeptides as they exist when outside the cell (SEQ ID NOS: 1, 2, 3, 4, 5, 6, 7, 8 or 9). Alternatively, the polypeptide of the invention may be made without a signal peptide sequence. Either way, the mature polypeptide does not possess the signal peptide sequence.

A signal peptide, comprised of about 10-30 hydrophobic amino acids, targets the nascent protein from the ribosome to the endoplasmic reticulum (ER). Once localized to the ER, the proteins can be further directed to the Golgi apparatus within the cell. The Golgi distributes proteins to vesicles, lysosomes, the cell membrane, and other organelles. Proteins targeted to the ER by a signal sequence can be released from the cell into the extracellular space. This is the case for the extracellular polypeptides of the present invention. For example, vesicles containing proteins to be moved outside the cell can fuse with the cell membrane and release their contents into the extracellular space via a process called exocytosis. Exocytosis can occur constitutively or after receipt of a triggering signal. In the latter case, the proteins are stored in secretory vesicles until exocytosis is triggered. Proteins that transit through this pathway are either released into the extracellular space or retained in the plasma membrane. Proteins that are retained in the plasma membrane contain one or more transmembrane domains, each comprised of about 20 hydrophobic amino acid residues. The polypeptides of the present invention lack both the transmembrane domain and the downstream cytoplasmic domain of the full-length protein.

The common structure of signal peptides from various proteins is typically described as a positively charged n-region, followed by a hydrophobic h-region and a neutral but polar c-region. The (−3, −1) rule states that the residues at positions −3 and −1 (relative to the signal peptide cleavage site) must be small and neutral for cleavage to occur correctly.

In many instances the amino acids comprising the signal peptide are cleaved off the protein during transport or once its final destination has been reached. Specialized enzymes, signal peptidases, are responsible for the removal of the signal peptide sequences from proteins. These enzymes are activated once the signal peptide has directed the protein to the desired location.

Polypeptides of the present invention may be produced recombinantly. In general, the signal sequence may be a component of an expression vector, or it may be a part of the DNA encoding the polypeptide of the invention that is inserted into such a vector. For E.coli expression, the signal sequence may be a prokaryotic signal sequence selected, for example, from the group of the alkaline phosphatase, penicillinase, 1pp, or heat-stable enterotoxin II leaders. For yeast secretion the signal sequence may be, e.g., the yeast invertase leader, alpha factor leader (including Saccharomyces and Kluyveromyces cc-factor leaders, the latter described in U.S. Pat. No. 5,010,182), or acid phosphatase leader, the C. albicans glucoamylase leader (EP 362,179), or the signal described in WO 90/13646. In mammalian cell expression, mammalian signal sequences may be used to direct secretion of the protein, such as signal sequences from secreted polypeptides of the same or related species as well as viral secretory leaders.

Fusion Polypeptides

The present invention embodies a method of using fusion proteins comprising purified and isolated, soluble receptor proteins IL20Rβs (SEQ ID NO. 2), IL20Rαs (SEQ ID NO. 1), LP338s (SEQ ID NO. 3), LP339s (SEQ ID NO. 4), LP340s (SEQ ID NO. 5), IL22Rs (SEQ ID NO. 6) and IL22RA2s (SEQ ID NO. 7) and IL22RA2 soluble splice variants named IL22BP1s (SEQ ID NO. 8) and IL22BP2s (SEQ ID NO. 9) and variants, complexes and combinations thereof to down-regulate IL19, IL20 and/or IL24 activity or combinations thereof.

The invention contemplates therapeutic utility for an isolated, individual soluble receptor subunit (purified protein with a sequence shown in SEQ ID NOS: 1, 2, 3, 4, 5, 6, 7, 8 or 9) and for compositions comprising mixtures of the isolated, individual receptor subunits. The invention further contemplates therapeutic utility for the isolated, individual soluble receptor subunits of the invention fused to a heterologous protein (e.g., an immunoglobulin constant region domain, an affinity tag) and compositions comprising mixtures thereof. Such fusion partners are preferably covalently joined to the carboxy terminus of the soluble receptor subunit. The invention further contemplates therapeutic utility for complexes of multiple, soluble receptor subunits (with or without a covalently attached heterologous fusion partner) wherein the multiple soluble receptor subunits are joined to each other, preferably by a peptide bond or by a disulfide bond or by a peptide linker. Their therapeutic utility is treatment and/or prevention of diseases involving the activity of IL10-related cytokines, further described below.

The present invention also embodies the method of using purified and isolated, soluble receptors comprised of a soluble IL20Rβ subunit and a soluble IL20Rα splice variant subunit, wherein the soluble IL20Rβ subunit is comprised of a polypeptide having an amino acid sequence selected from the group consisting of SEQ ID No. 2, variants thereof and fusion proteins comprising SEQ ID No. 2 and variants thereof, and the IL20Rα splice variant subunit is comprised of a polypeptide having an amino acid sequence selected from the group consisting of SEQ ID NOs: 3, 4, 5, variants thereof and fusion proteins comprising SEQ ID NOs: 3, 4, 5, or variants thereof, to downregulate IL20, IL19 and/or IL24 activity or any combination thereof in vivo, in situ, or in vitro.

The soluble IL20Rβ subunit and the soluble IL20Rα splice variant subunit are preferably linked together. The linking between soluble IL20Rβ subunit and soluble IL20Rα splice variant subunit can be by any means, but preferably by a peptide bond or a disulfide bond between a heterologous polypeptide fusion partner covalently linked to the soluble IL20Rβ subunit and a heterologous polypeptide fusion partner covalently linked to the soluble IL20Rα splice variant subunit. Linking can also be accomplished by means of a polypeptide linker. The polypeptide linker should be between about 20-300 amino acid residues in length, preferably about 150-200 amino acid residues in length. A suitable linker would be comprised of glycine and serine residues. Another suitable linker would be a random sequence devoid of cysteine and proline residues. Another suitable linker would be multiple units of Gly-Gly-Gly-Gly-Ser. The polypeptide linker would preferably extend from the amino terminus of one receptor subunit extracellular domain to the carboxyl terminus of the extracellular domain of the other receptor subunit. Alternatively the polypeptide linker could extend from a fusion partner of one soluble receptor subunit to the fusion partner of another soluble receptor subunit. These soluble receptors and soluble receptor complexes are useful for the downregulation of IL20, IL19 and/or IL24 activity or any combination thereof in vivo, in situ, and in vitro.

The present invention embodies a method of using purified and isolated, soluble receptors comprised of a soluble IL20Rβ subunit and a soluble IL20Rα subunit, wherein the soluble IL20Rβ subunit is comprised of a polypeptide having an amino acid sequence selected from the group consisting of SEQ ID No. 2, variants thereof and fusion proteins comprising SEQ ID No. 2 and the soluble IL20Rα subunit is comprised of a polypeptide having an amino acid sequence selected from the group consisting of SEQ ID No: 1, variants thereof and fusion proteins comprising SEQ ID No: 1, for the downregulation of IL20, IL19 and/or IL24 activity or any combination thereof in vivo, in situ, or in vitro.

The soluble IL20Rβ subunit and the soluble IL20Rα subunit are preferably linked together by a polypeptide linker. The linking between a soluble IL20Rβ subunit and a soluble IL20Rα subunit can be by any means, but preferably by a peptide bond or a disulfide bond between a heterologous polypeptide fusion partner covalently linked to the soluble IL20Rβ subunit and a heterologous polypeptide fusion partner covalently linked to the IL20Rα subunit. The linking can also be by a polypeptide linker. The polypeptide linker should be between about 20-300 amino acid residues in length, preferably about 150-200 amino acid residues in length. A suitable linker would be comprised of glycine and serine residues. Another suitable linker would be a random sequence devoid of cysteine and proline residues. Another suitable linker would be multiple units of Gly-Gly-Gly-Gly-Ser. The polypeptide linker would preferably extend from the amino terminus of one soluble receptor subunit to the carboxyl terminus of the other soluble receptor subunit. Alternatively the polypeptide linker could extend from a fusion partner of one soluble receptor subunit to the fusion partner of another soluble receptor subunit. These soluble receptors and soluble receptor complexes are useful for the downregulation of IL20, IL19 and/or IL24 activity or any combination thereof.

The present invention also embodies purified and isolated, soluble receptors comprised of a soluble IL20Rβ subunit and a soluble IL22R subunit or a soluble IL22RA2 subunit or soluble IL22BP1 subunit or soluble IL22BP2 subunit, wherein the soluble IL20Rβ subunit is comprised of a polypeptide selected from the group consisting of a polypeptide with a sequence shown in SEQ ID No. 2, variants thereof, and fusion proteins comprising a polypeptide with the sequence shown in SEQ ID NO. 2 and the soluble IL22R subunit is comprised of a polypeptide selected from the group consisting of a polypeptide with the sequence shown in SEQ ID No. 6, variants therof and fusion proteins comprising a polypeptide with the sequence shown in SEQ ID No. 6 and the soluble IL22RA2 subunit is comprised of a polypeptide selected from the group consisting of SEQ ID No. 7, variants thereof and fusion proteins comprising SEQ ID No. 7 and the soluble IL22BP1 subunit is comprised of polypeptide selected from the group consisting of SEQ ID No. 8, variants thereof and fusion proteins comprising SEQ ID No. 8 and the soluble IL22BP2 subunit is comprised of polypeptide selected from the group consisting of SEQ ID NO. 9, variants thereof and fusion proteins comprising SEQ ID No. 9.

The IL20Rβs subunit and the IL22Rs or IL22RA2s or IL22BP1s or IL22BP2s subunit are preferably linked together by a polypeptide linker. Linking between IL20Rβs subunit and IL22Rs or IL22RA2s or IL22BP1s or IL22BP2s subunit can be by any means, but preferably by a peptide bond or a disulfide bond between a heterologous polypeptide covalently linked to the IL20Rβs subunit and a heterologous polypeptide covalently linked to the IL22Rs or IL22RA2s or IL22BP1s or IL22BP2s subunit. Linking can also be by a polypeptide linker. The polypeptide linker should be between about 20-300 amino acid residues in length, preferably about 150-200 amino acid residues in length. A suitable linker would be comprised of glycine and serine residues. Another suitable linker would be a random sequence devoid of cysteine and proline residues. Another suitable linker would be multiple units of Gly-Gly-Gly-Gly-Ser. The polypeptide linker would preferably extend from the amino terminus of one subunit extracellular domain to the carboxyl terminus of the extracellular domain of the other subunit. Alternatively the polypeptide linker could extend from a fusion polypeptide of one soluble receptor subunit to the fusion partner of another aoluble receptor subunit.

One aspect of the invention provides an isolated and purified fusion polypeptide consisting essentially of a first portion and a second portion joined by a third portion. The first portion of the fusion polypeptide comprises (a) a protein with a sequence shown in SEQ ID Nos. 1, 2, 3, 4, 5, 6, 7, 8 or 9 (b) a polypeptide that has an amino acid sequence at least 90%, preferably at least 91%, 92%, 93% or 94%, more preferably at least 95%, even more preferably at least 96%, 97%, or 98% and most preferably at least 99% identical (i.e., amino acid sequence identity) to that described in (a). The second portion of the fusion polypeptide consists of another polypeptide. Within one such embodiment the fusion partner (i.e., the second portion) is a constant region of the heavy chain of an immunoglobulin (Ig) molecule or a portion thereof or the constant region of the light chain of an Ig molecule. Within another embodiment the fusion partner (i.e., the second portion) is an affinity tag (e.g. FLAG or His6 or an immunoglobulin Fc polypeptide) or a reporter molecule (e.g. luciferase or beta-galactosidase). Within yet another emodiment, the fusion partner (i.e., the second portion) comprises (a) a protein with a sequence shown in SEQ ID Nos. 1, 2, 3, 4, 5, 6, 7, 8 or 9 (b) a protein polypeptide that has an amino acid sequence at least 90%, preferably at least 91%, 92%, 93% or 94%, more preferably at least 95%, even more preferably at least 96%, 97%, or 98% and most preferably at least 99% identical (i.e., amino acid sequence identity) to that described in (a). The third portion, separating the first portion and the second portion of the fusion protein, is selected from the group consisting of a peptide bond, a disulfide bond or a polypeptide linker sequence. The polypeptide linker is preferably between about 20-300 amino acid residues in length, more preferably about 150-200 amino acid residues in length. A suitable linker would be comprised of glycine and serine residues. Another suitable linker would be a random sequence devoid of cysteine and proline residues. Another suitable linker would be multiple units of Gly-Gly-Gly-Gly-Ser.

In one preferred embodiment one extracellular subunit polypeptide has a constant region of a heavy chain of an immunoglobulin (Ig) fused to its carboxy terminus and the other extracellular subunit has a constant light chain of an immunoglobulin fused to its carboxy terminus such that the two polypeptides come together to form a soluble receptor and a disulfide bond is formed between the heavy and the light Ig chains. In another preferred embodiment one extracellular subunit polypeptide has a constant region of a heavy chain of an immunoglobulin (Ig) fused to its carboxy terminus and the other extracellular subunit has a heavy chain constant region (preferably the same type as fused to the other subunit) of an immunoglobulin fused to its carboxy terminus such that the two polypeptides come together to form a soluble receptor and a disulfide bond is formed between the heavy and the light Ig chains. In another preferred embodiment, a peptide linker could be fused to the two carboxy-termini of the polypeptides to form a covalently bonded soluble receptor.

The invention further contemplates fusion polypeptides, two or more, being linked together to form a “complex” of fusion polypeptides. The fusion polypeptides and complexes of fusion polypeptides of the invention are useful for the method of downregulating IL20, IL19 and/or IL24 activity or any combination thereof.

Polypeptide Variants

Reference to a particular soluble polypeptide sequence disclosed in SEQ ID NOS: 1, 2, 3, 4, 5, 6, 7, 8 or 9 (i.e., “polypeptide of the invention”) as well as fusion proteins comprising polypeptides of the invention is also understood to include variants of the polypeptide as defined herein. The term “variant” refers to a polypeptide differing from a polypeptide of the present invention, but retaining essential properties thereof. Generally, variants are closely similar overall in structural and/or sequence identity, and, in many regions, identical to polypeptide of the present invention.

The present invention is also directed to polypeptides that comprise, or alternatively consist of, an amino acid sequence that is at least: 90%, 95%, 96%, 97%, 98%, 99% identical to a polypeptide sequence of SEQ ID NOs: 1, 2, 3, 4, 5, 6, 7, 8 or 9 or fusion proteins comprising a polypeptide sequence of SEQ ID NOs: 1, 2, 3, 4, 5, 6, 7, 8 or 9

A polypeptide exhibiting or having at least about, e.g., 95% “sequence identity” to another amino acid sequence may include, e.g., up to five amino acid alterations per each 100 amino acid (on average) stretch of the test amino acid sequence. In other words, a first amino acid sequence that is at least 95% identical to a second amino acid sequence, can have up to 5% of its total number of amino acid residues different from the second sequence, e.g., by insertion, deletion, or substitution of an amino acid residue.

Alterations in amino residues of a polypeptide sequence may occur at the amino or carboxy terminal positions or anywhere between these terminal positions, interspersed either individually among residues in the sequence or in one or more contiguous fragments within the sequence. As a practical matter, whether any particular polypeptide sequence exhibits at least about: 90%, 91%, 92%, 93%, 94% 95%, 96%, 97%, 98%, or 99% similarity to another sequence, can be determined conventionally by using known methods in the art.

The phrase “percent (%) identity” with respect to the amino acid sequences identified herein is defined as the percentage of amino acid residues in a candidate sequence that are identical with the amino acid residues in a reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as ALIGN, ALIGN-2, Megalign (DNASTAR) or BLAST (e.g., Blast, Blast-2, WU-Blast-2) software. Those skilled in the art can determine appropriate parameters for measuring alignment, including any algorithms needed to achieve maximal alignment over the full length of the sequences being compared. For example, the % identity values used herein are generated using WU-BLAST-2 [Altschul, et al., Methods in Enzymology 266: 460-80 (1996)]. Most of the WU-BLAST-2 search parameters are set to the default values. Those not set to default values, i.e., the adjustable parameters, are set with the following values: overlap span=1; overlap fraction=0.125; word threshold (T)=11; and scoring matrix=BLOSUM 62. For purposes herein, a % amino acid sequence identity value is determined by dividing (a) the number of matching identical amino acid residues between the amino acid sequence of the hSEZ6 polypeptide of interest and the comparison amino acid sequence of interest (i.e., the sequence against which the hSEZ6 polypeptide of interest is being compared) as determined by WU-BLAST-2, by (b) the total number of amino acid residues of the polypeptide of interest.

Variants may be produced by mutagenesis techniques or by direct synthesis using known methods of protein engineering and recombinant DNA technology. Such variants may be generated to improve or alter the characteristics of the polypeptide or the expression levels or may occur unintentionally. One or more amino acids can often be deleted from the N-terminus or C-terminus of a secreted polypeptide without a substantial loss of biological function. Moreover, ample evidence demonstrates that polypeptide variants can retain a biological activity similar to that of the naturally occurring protein. Even if deleting one or more amino acids from the N-terminus or C-terminus of the polypeptide results in modification or loss of one or more biological functions, other biological activities may be retained.

Variants of the polypeptides of the invention can be generated through DNA shuffling as disclosed, for example, by International Patent Application WO 97/20078 and U.S. Pat. Nos. 6,303,344 and 6,297,053.

The invention also encompasses polypeptide variants that show a biological activity of the reference polypeptide such as, e.g., ligand binding or antigenicity. Such variants include, e.g., deletions, insertions, inversions, repeats, and substitutions selected so as to have little effect on activity using general rules known in the art. For example, teachings on making phenotypically silent amino acid substitutions are provided (15).

One technique compares amino acid sequences in different species to identify the positions of conserved amino acid residues since changes in an amino acid at these positions are more likely to affect a protein function. In contrast, the positions of residues where substitutions exist more frequent generally indicate that amino acid residues at these positions are less critical for a protein function. Thus positions tolerating amino acid substitutions typically may be modified while still maintaining a biological activity of a protein.

Another technique uses genetic engineering to introduce amino acid changes at specific positions of a polypeptide to identify regions critical for a protein function. For example, site directed mutagenesis or alanine-scanning mutagenesis (the introduction of single alanine mutations at every residue in the molecule) can be used (16). A resulting mutant can subsequently be tested for a biological activity.

These two techniques have revealed that proteins are surprisingly tolerant of amino acid substitutions and they generally indicate which amino acid changes are likely to be permissive at certain amino acid positions in a protein. For example, typically, most buried amino acid residues (those within the tertiary structure of the protein) require nonpolar side chains, whereas few features of surface side chains are generally conserved. Preferred conservative amino acid substitutions are listed in Table 1 herein.

Polypeptide variants containing amino acid substitutions of charged amino acids with other charged or neutral amino acids may produce polypeptides with improved characteristics e.g., such as less aggregation. Aggregation of pharmaceutical formulations both reduces activity and increases clearance due to the aggregate's immunogenic activity (17).

A further embodiment of the invention encompasses a protein that comprises an amino acid sequence of the present invention that contains at least one amino acid substitution, but not more than 20 amino acid substitutions, preferably not more than 15 amino acid substitutions.

Of course, in order of ever-increasing preference, it is highly preferable for a polypeptide of the invention to have an amino acid sequence that comprises an amino acid sequence of the present invention which contains zero or one, but not more than: 10, 9, 8, 7, 6, 5, 4, 3, 2, or 1 amino acid substitutions; wherein conservative amino acid substitutions are more preferable than non-conservative substitutions

Modifications of Soluble Cytokine Receptor Polypeptides

The polypeptides of the invention are composed of amino acids joined to each other by peptide bonds or modified peptide bonds, i.e., peptide isosteres, and may contain amino acids other than the gene-encoded amino acids. The polypeptides of the invention may be modified by either natural processes, such as posttranslational processing, or by chemical modification techniques which are well known in the art. Modifications can occur anywhere in the polypeptides, including peptide backbone, amino acid side-chains and amino or carboxyl termini. The same type of modification may be present in the same or varying degrees at several sites in a given polypeptide of the invention. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, reference 18-21).

A type of covalent modification of the polypeptides of the present invention included within the scope of this invention comprises altering the native glycosylation pattern of the polypeptide. “Altering the native glycosylation pattern” is intended for purposes herein to mean deleting one or more carbohydrate moieties found in native sequence polypeptide and/or adding one or more glycosylation sites that are not present in the native sequences. Additionally, the phrase includes qualitative changes in the glycosylation of the native proteins, involving a change in the nature and proportions of the various carbohydrate moieties present.

Addition of glycosylation sites to polypeptides may be accomplished by altering the amino acid sequence thereof. The alteration may be made, for example, by the addition of, or substitution by, one or more serine or threonine residues to the native sequence (for O-linked glycosylation sites). The amino acid sequences may optionally be altered through changes at the DNA level, particularly by mutating the DNA encoding the polypeptides at preselected bases such that codons are generated that will translate into the desired amino acids.

Another means of increasing the number of carbohydrate moieties on the polypeptides is by chemical or enzymatic coupling of glycosides to the polypeptide. Such methods are described in the art, e.g., in WO 87/05330, and in reference 22.

Removal of carbohydrate moieties present on the polypeptide may be accomplished chemically or enzymatically or by mutational substitution of codons encoding for amino acid residues that serve as targets for glycosylation. Chemical deglycosylation techniques are known in the art. Enzymatic cleavage of carbohydrate moieties on polypeptides can be achieved by the use of a variety of endo- and exo-glycosidases (23).

Another type of covalent modification of polypeptides comprises linking any one of the polypeptides to one of a variety of nonproteinaceous 20 polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Expression of Soluble Cytokine Receptor Polypeptides

Nucleic acid encoding a polypeptide of the invention may be obtained from a cDNA library prepared from tissue believed to possess the mRNA encoding the polypeptide and to express it at a detectable level. The nucleic acid preferably encodes the signal peptide sequence at the amino terminus of the polypeptide. The nucleic acid is lacking the sequence encoding the transmembrane and cytoplasmic domains of the full-length polypeptide. Libraries can be screened with probes (such as antibodies to a polypeptide or oligonucleotides of at least about 20-80 bases) designed to identify the gene of interest or the protein encoded by it. Screening the cDNA or genomic library with the selected probe may be conducted using standard procedures, such as those described in references 10 and 30. An alternative means to isolate the gene encoding a polypeptide of the invention is to use PCR methodology (10). Further details of the cloning and expression of the polypeptides of the invention are in the Examples herein.

Recombinant expression vectors are typically self-replicating DNA or RNA constructs containing a desired gene to be expressed operably linked to a promoter and optionally other control elements recognized in a suitable host cell. The specific type of control elements necessary to effect expression depends on the host cell used and the level of expression desired. Proteins can be expressed in mammalian cells, yeast, bacteria, or other cells under the control of appropriate promoters.

Vectors, as used herein, encompass plasmids, viruses, bacteriophage, integratable DNA fragments, and other vehicles that enable the integration of DNA fragments into the genome of the host although, optionally, expression can occur transiently without integration. Plasmids are the most commonly used form of vector, but many other forms of vectors that perform an equivalent function are also suitable for use (24).

Both expression vectors and cloning vectors contain a nucleic acid sequence that enables the vector to replicate in one or more selected host cells. Such sequences are well known for a variety of bacteria, yeast, and viruses.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement autotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the polypeptide-encoding nucleic acid, such as DHFR or thymidine kinase. An appropriate host cell, when wild-type DHFR is employed, is the CHO cell line deficient in DHFR activity (25). A suitable selection gene for use in yeast is the trp1 gene present in the yeast plasmid YRp7 (26). The trp1 gene provides a selection marker for a mutant strain of yeast lacking the ability to grow in tryptophan, for example, ATCC No. 44076.

Expression vectors contain a promoter operably linked to the polypeptide-encoding nucleic acid sequence to direct mRNA synthesis. Promoters recognized by a variety of potential host cells are well known. Promoters for use in bacterial systems also will contain a Shine-Dalgarno sequence operably linked to the DNA encoding a polypeptide of interest.

Transcription of a DNA encoding a polypeptide by higher eukaryotes may be increased by inserting an enhancer sequence into the vector. Enhancers are cis-acting elements of DNA, usually about from 10 to 300 bp, that act on a promoter to increase its transcription level. Many enhancer sequences are known from mammalian genes (globin, elastase, albumin, a-ketoprotein, and insulin). Typically, however, one will use an enhancer from a eukaryotic-cell virus. Examples include the SV40 enhancer on the late side of the replication origin (bp 100-270). The enhancer may be spliced into the vector at a position 5′ or 3′ to the polypeptide coding sequence.

Expression vectors used in eukaryotic host cells will also contain sequences necessary for the termination of transcription and optionally for stabilizing the mRNA. Such sequences are commonly available from the 5′ and occasionally 3′ untranslated regions of eukaryotic or viral DNAs or cDNAs. These regions contain nucleotide segments transcribed as polyadenylated fragments in the untranslated portion of the mRNA encoding an LP polypeptide.

Expression in Host Cells

The description below relates primarily to production of polypeptides of the invention by culturing cells transformed or transfected with a vector containing polypeptide-encoding nucleic acid operably linked to expression elements. It is contemplated that alternative methods, which are well known in the art, may be employed to prepare polypeptides of the invention. For instance, the polypeptide sequence, or portions thereof, may be produced by direct peptide synthesis using solid-phase techniques well-known in the art. In vitro protein synthesis may be performed using manual techniques or by automation. Automated synthesis may be accomplished, for instance, using an Applied Biosystems Peptide Synthesizer (Foster City, Calif.) and manufacturer's instructions. Various portions of a polypeptide of the invention may be chemically synthesized separately and combined using chemical or enzymatic methods to produce a full-length polypeptide.

Host cells are transfected or transformed with expression vectors or cloning vectors described herein for polypeptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: A Practical Approach, M. Butler, ed. (IRL Press, 1991) and Sambrook el al., supra. Methods of transfection are known to the ordinarily skilled artisan, for example, CaPO₄ and electroporation.

Suitable host cells for cloning or expressing the nucleic acid (e.g., DNA) in the vectors herein include prokaryote, yeast, or higher eukaryote cells. Suitable prokaryotes include but are not limited to E. coli K12 strain MM294 (ATCC 3 1.446); E. coli X1 776 (ATCC 3 1.537); E. coli strain W3110 (ATCC 27.325) and K5 772 (ATCC 53.635). Other suitable prokaryotic host cells include Enterobacter, Erwinia, Klebisella, Proteus, Salmonella, Serratia, and Shigeila, as well as Bacilli, Pseudomona, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. Alternatively, in vivo methods of cloning, e.g., PCR or other nucleic acid polymerase reactions, are suitable.

In addition to prokaryotes, eukaryotic microbes such as filamentous fungi or yeast are suitable cloning or expression hosts for LP polypeptide expressing vectors. Saccharomyces cerevisiae is a commonly used lower eukaryotic host microorganism. Many others are used by those in the art.

Suitable host cells for the expression of glycosylated polypeptides of the invention are derived from multicellular organisms. Examples of invertebrate cells include insect cells such as Drosophila S2 and Spodoptera Sp, Spodoptera high5 as well as plant cells. Examples of useful mammalian host cell lines include, but are not limited to, EBNA-293, CHO and COS cells. The selection of the appropriate host cell is within the skill in the art.

Polypeptide Purification

It is preferred to purify the polypeptides of the present invention to greater than about 80% purity, more preferably to greater than about 90% purity, even more preferably greater than about 95% purity, and particularly preferred is a pharmaceutically pure state, that is greater than about 99.9% pure with respect to contaminating macromolecules, particularly other proteins and nucleic acids, and free of infectious and pyrogenic agents. Preferably a purified polypeptide is substantially free of other polypeptides, particularly other polypeptides of animal origin.

Gene expression may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal and may be prepared in any mammal. Conveniently, the antibodies may be prepared against a native sequence provided herein or against exogenous sequence fused to a polypeptide-encoding DNA and encoding a specific antibody epitope.

Forms of polypeptides may be recovered from culture medium or from host cell lysates. The polypeptides of the present invention are not membrane-bound. Cells employed in expression of polypeptides can be disrupted by various physical or chemical means, such as freeze-thaw cycling, sonication, mechanical disruption, or cell lysing agents.

The following procedures are exemplary of suitable purification procedures for expressed recombinant polypeptides or chimeric polypeptides: fractionation on an ion-exchange column; ethanol precipitation; acid or chaotrope extraction, reversed-phase HPLC; FPLC, hydroxyapatite size exclusion, chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of polypeptides. Various methods of protein purification may be employed and such methods are known in the art and described, for example, in reference 28. The purification step(s) selected will depend, for example, on the nature of the production process used and the particular polypeptide produced. Purification of the polypeptides of the invention are futher described in the Examples herein.

Polypeptide Analysis

Many types of analyses can be performed with the polypeptides of the invention to demonstrate their role in the development, pathogenesis, and treatment of cancer, cardiovascular disease and immune related disease, e.g., inflammation. Certain analyses are exemplified in the Examples herein. Protocols for the analyses may be found in references 10 and 30 and others referenced herein.

Cell-based assays using a cell type (optionally known to be involved in a particular disease) are transfected with one or more vectors expressing a polypeptide of the invention. Such cells are monitored for phenotypic changes, for example T-cell proliferation by mixed lymphocyte reaction, inflammatory cell infiltration, cytokine levels, ligand binding and reaction to particular antibodies. While transiently-transfected cells can be used, stable cell lines expressing soluble polypeptides of the invention are preferred.

Alternatively, as described in Example 8, one or more purified polypeptides of the invention may be added along with varying amounts of a cytokine of interest to cells expressing a normal receptor for that particular cytokine.

Uses

The invention provides a method of modulating the physiology or development of a cell in vivo or in situ comprising introducing into such cell, or the environment of such cell, a therapeutically effective amount of a composition comprising purified protein with an amino acid sequence as represented by SEQ ID NOs: 1, 2, 3, 4, 5, 6, 7, 8, 9 or a variant, combination, or complex thereof or a purified fusion protein comprising a protein with amino acid sequence shown in SEQ ID NOs: 1, 2, 3, 4, 5, 6, 7, 8, 9, or a variant, combination, or complex thereof for the purpose of treating or preventing diseases that involve the binding of an IL10-related cytokine to its normal receptor. The invention thereby inhibits or decreases the normal IL10-related cytokine signal-transduction pathway. Numerous such diseases or disorders exist including, but not limited to, inflammation, immune system, cardiovascular and hematopoietic disorders and regulation of cellular proliferation.

Purified polypeptides whose sequence is shown in SEQ ID NOs 1, 2, 3, 4, 5, 6, 7, 8, 9 and variants, complexes and combinations particularly those exemplified in Table 2a herein) thereof and purified fusion proteins comprising a protein with a sequence shown in SEQ ID NOs: 1, 2, 3, 4, 5, 6, 7, 8, 9 and variants, combinations (particularly those exemplified in Table 2a herein) and complexes thereof are useful for the prevention and treatment of diseases in which IL10-related cytokines are involved, including, but not limited to, inflammation, immune system, cardiovascular and hematopoietic disorders and regulation of cellular proliferation. These purified reagents can be administered to a patient, veterinary or human.

Particular cancers suitable for treatment with the polypeptides of the invention or variants, combinations, or complexes thereof or fusion proteins comprising the polypeptides of the invention or variants combinations or complexes thereof include, but are not limited to, acute myelogenous leukemias including acute monocytic leukemia, acute myeloblastic leukemia, acute promyelocytic leukemia, acute myelomonocytic leukemia, acute erythroleukemia, acute megakaryocyticleukemia, and acute undifferentiated leukemia, etc.; and chronic myelogenous leukemias including chronic myelomonocytic leukemia and chronic granulocyticleukemia, adenocarcinoma, lymphoma, melanoma, myeloma, Hamartoma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus and also angiogenesis.

Particular cardiovascular disorder suitable for treatment with the polypeptides of the invention or variants, combinations, or complexes thereof or fusion proteins comprising the polypeptides of the invention or variants combinations or complexes thereof include, but are not limited to, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, complications of cardiac transplantation, arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery.

Particular immune system disorders suitable for treatment with the polypeptides of the invention or variants, combinations, or complexes thereof or fusion proteins comprising the polypeptides of the invention or variants combinations or complexes thereof include, but are not limited to, inflammatory disorders, acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, cystic fibrosis, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, inflammatory lung disease, irritable bowel syndrome, multiple sclerosis, myasthenia gravis, myocardial or pericardial inflammation, multiple organ failure, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, septic shock, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma.

Hematopoietic disorders suitable for treatment with the polypeptides of the invention or variants, combinations, or complexes thereof or fusion proteins comprising the polypeptides of the invention or variants combinations or complexes thereof include, but are not limited to, disorders requiring the repopulation of red blood cells, granulocytes, platelets, and/or monocytes, surgery, anemia, CML, thrombocytopenia, myeloproliferative disorders, renal disease, dialysis, hemorrhage, leukemia.

Therapeutic formulations are prepared for storage by optionally mixing the active ingredient having the desired degree of purity with pharmaceutically acceptable carriers, excipients or stabilizers [Remington's Pharmaceutical Sciences 16th edition (1980)], in the form of lyophilized formulations or aqueous solutions.

The formulation herein may also contain more than one active compound as necessary for the particular indication being treated, preferably those with complementary activities that do not adversely affect each other. Such molecules are suitably present in combination in amounts that are effective for the purpose intended.

The active agents of the present invention are administered to a mammal, preferably a human, in accord with known methods, such as intravenous administration as a bolus or by continuous infusion over a period of time, by intramuscular, intraperitoneal, intracerebral, intracerobrospinal, subcutaneous, intra-articular, intrasynovial, intrathecal, intraoccular, intralesional, oral, topical, inhalation, pulmonary, and/or through sustained release.

Other therapeutic regimens may be combined with the administration of a polypeptide of the invention. For the prevention or treatment of disease, the appropriate dosage of an active agent will depend on the type of disease to be treated, as defined above, the severity and course of the disease, whether the agent is administered for preventive or therapeutic purposes, previous therapy, the patient's clinical history and response to the agent, and the discretion of the attending physician. The agent is suitably administered to the patient at one time or over a series of treatments.

Dosages and desired drug concentration of pharmaceutical compositions of the present invention may vary depending on the particular use envisioned. The determination of the appropriate dosage or route of administration is well within the skill of an ordinary artisan and is dependent upon many factors including means of administration, target site, physiological state of the patient, and other medications administered. Thus, treatment dosages should be titrated to optimize safety and efficacy. Animal experiments provide reliable guidance for the determination of effective does for human therapy. Interspecies scaling of effective doses can be performed following the principles laid down by Mordenti and Chappell, “The Use of Interspecies Scaling in Toxicokinetics,” in Toxicokinetics and New Drug Development, Yacobi et al., Eds., Pergamon Press, NY 1989, pp.4246.

When in vivo administration of a polypeptide of the invention is employed, normal dosage amounts may vary from about 1 ng/kg up to 100 mg/kg of mammal body weight or more per day, preferably about 1 pg/kg/day up to 100 mg/kg of mammal body weight or more per day, depending upon the route of administration. Guidance as to particular dosages and methods of delivery is provided in the literature; see, for example, U.S. Pat. Nos. 4,657,760, 5,206,344 or 5,225,212. It is within the scope of the invention that different formulations will be effective for different treatment compounds and different disorders, that administration targeting one organ or tissue, for example, may necessitate delivery in a manner different from that to another organ or tissue. Moreover, dosages may be administered by one or more separate administrations or by continuous infusion. For repeated administrations over several days or longer, depending on the condition, the treatment is sustained until a desired suppression of disease symptoms occurs. However, other dosage regimens may be useful. The progress of this therapy is readily monitored by conventional techniques and assays.

The invention is further illustrated by the following non-limiting examples.

### EXAMPLES

General Methods

Commercially available reagents referred to in the examples are used according to manufacturer's instructions unless otherwise indicated. The source of cells identified in the following examples, and throughout the specification, by ATCC accession numbers is the American Type Culture Collection, Manassas, Va. Unless otherwise noted, the present invention uses standard procedures of recombinant DNA technology such as those described or referenced in references 10 and 30.

### Example 1

### Cloning into a Mammalian Expression Vector

Vectors encoding Flag-HIS (FLIS)-tagged polypeptides of the invention are transfected into an approproiate mammalian cell line for expression (e.g., HEK-293EBNA, Cos-7 (ATCC CRL-1651) or HEK-293T or CHO) in order to generate enough recombinant protein for study. For example, the human LP154 is engineered for expression as follows.

Many different PCR primers for expression of each polypeptide of interest may be designed by one of skill in the art. For example, for LP154, the PCR oligonucleotide primers may be 5′-ccgccgggcgcgccaccatgcagactttcacaatggt-3′ (SEQ ID NO. 17) and 5′-ccgccggatatctccttgcacctccacacatt-3′ (SEQ ID NO. 18) for the forward and reverse strands, respectively. For LP347, the PCR oligonucleotide primers may be 5′-ccgccggcgcgccaccatgcgggctcccggccgccc-3′ (SEQ ID NO. 19) and 5′-ccgccgatatcttttagccttgaactctgatg-3′ (SEQ ID NO. 20). For IL22RA2, the PCR primers may be 5′-agctgccttcttcacttg-3′ (SEQ ID NO. 21) and 5′ttgctctgcctcttattc-3′ (SEQ ID NO. 22) (see ref. 42). Design of PCR primers is well within the normal skill of a molcular biologist and can be based on published sequences who sources are previously described herein. The resultant PCR generated fragment is purified, cleaved with appropriate restriction enzyme(s), again purified and ligated into the mammalian expression vector pEW1969 (a derivative of pJB02, Eli Lilly) that is digested with compatible restriction enzyme(s). Alternative mamalian expression vectors can be used in place of pEW1969. For example, the pINCY vector (Incyte Genomics, Palo Alto, Calif.) or other alternative vectors are available from multiple companies including Promega Corp. (Madison, Wis.) and Clontech (Palo Alto, Calif.). Preferably the expression vector has a tag, such as FLAG-His, suitable for use in purifying the expressed protein.

One of skill in the art knows how to adapt this method, or alternative methods cited in references hereinabove, for construction of mammalian expression vectors containing polynucleotide sequence encoding a polypeptide with a sequence shown in SEQ ID NOS: 1, 2, 3, 4, 5, 6, 7, 8 or 9 either with or without a polynucleotide sequence encoding the corresponding amino-terminal signal peptide signal.

HT-FlagHIS Protein Production

The vectors are separately transiently transfected into mammalian cells (e.g., HEK-293, COS-7 or HEK-293T or CHO cells) (31) using FUGENE as described by the vendor (Roche Molecular Bioproducts). The recombinant proteins are harvested and measured in supernatants collected from the transfected cells by Western-blot using anti-Flag antibody and Coomassie-stained analyses.

### Example 2

### Expression and Purification of Polypeptides of Interest in E. coli

The bacterial expression vector pQE60 is used for bacterial expression in this example although other bacterial expression vectors are commercially available (QIAGEN, Inc., Chatsworth, Calif.). pQE60 encodes an ampicillin antibiotic resistance gene (Amp^{r}) and contains a bacterial origin of replication (ori), an IPTG-inducible promoter, a ribosome binding site (RBS), six codons encoding histidine (His6 tag) residues that allow affinity purification using nickel-nitrilo-tri-acetic acid (Ni-NTA) affinity resin sold by QIAGEN, Inc., and single restriction enzyme cleavage sites suitable for cloning (i.e., in a multiple cloning site). These elements are arranged such that a DNA fragment encoding a polypeptide of interest can be operably linked in such a way as to produce that polypeptide with the six His residues covalently linked to the carboxyl terminus of that polypeptide. However, a polypeptide coding sequence can optionally be inserted in such a way that translation of the six His codons is prevented and, therefore, a polypeptide is produced with no 6×His tag.

The nucleic acid sequence encoding the desired portion LP347, LP154, LP338, LP339, LP340, IL22R, IL22RA2, IL22BP1s and IL22BP2s either with or without the nucleotide sequence encoding the leader sequence is amplified from a cDNA clone using PCR oligonucleotide primers, which anneal, one upstream (or 5′) and one downstream (or 3′), to the desired portion of the polypeptide. Additional nucleotides containing restriction sites to facilitate cloning in the pQE60 vector may be added to the 5′ and 3′ sequences, respectively.

The amplified nucleic acid fragments (e.g., those described in Example 1 hereinabove) and the vector pQE60 are digested with appropriate restriction enzymes and the digested DNAs are then ligated together. Insertion of the polypeptide-encoded DNA into the restricted pQE60 vector places the polypeptide coding region including its associated stop codon downstream from the IPTG-inducible promoter and operably linked in-frame with an initiating AUG codon. The associated stop codon prevents translation of the six histidine codons downstream of the insertion point.

The ligation mixture is transformed into competent E. coli cells using standard procedures. E. coli strain M15/rep4, containing multiple copies of the plasmid pREP4, which expresses the lac repressor and confers kanamycin resistance (Kan^{r}), is used in carrying out the illustrative example described herein. This strain, which is only one of many that are suitable for expressing polypeptides, is available commercially from QIAGEN, Inc. Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Bacteria containing the desired cloned constructs are grown overnight (O/N) in liquid culture in LB media (Sigma Corp. St. Louis, Mo.) supplemented with both ampicillin (100 μg/ml) and kanamycin (25 μg/ml). The O/N culture is used to inoculate a large culture, at a dilution of approximately 1:25 to 1:250. The cells are grown to an optical density at 600 nm (OD600) of between 0.4 and 0.6. Isopropyl-b-D-thiogalactopyranoside (IPTG) is then added to a final concentration of 1 mM to induce transcription from the lac repressor sensitive promoter, by inactivating the lacI repressor. Cells subsequently are incubated further for 3 to 4 hours. Cells then are harvested by centrifugation.

The cells are then stirred for 3-4 hours at 4° C. in 6 M guanidine-HCl, pH 8.0. The cell debris is removed by centrifugation, and the supernatant containing the LP polypeptide is dialyzed against 50 mM Na-acetate buffer pH 6.0, supplemented with 200 mM NaCl. Alternatively, a polypeptide can be successfully refolded by dialyzing it against 500 mM NaCl, 20% glycerol, 25 mM Tris/HCl pH 7.4, containing protease inhibitors.

If insoluble protein is generated, the protein is made soluble according to known method steps. After renaturation, the polypeptide is purified for example by ion exchange, hydrophobic interaction, and size exclusion chromatography. Alternatively, an affinity chromatography step such as an antibody column is used to obtain purified polypeptide. The purified polypeptide is stored at 4° C. or frozen at −40° C. to −120° C.

### Example 3

### Cloning and Expression in a Baculovirus System

In this example, the plasmid shuttle vector pA2 GP is used to insert the cloned DNA encoding the IL22Rs polypeptide (SEQ ID NO. 12), without the sequence encoding the signal peptide, into a baculovirus to express the polypeptide, using a baculovirus leader and standard methods as described in Summers, et al., A Manual of Methods for Baculovirus Vectors and Insect Cell Culture Procedures, Texas Agricultural Experimental Station Bulletin No. 1555 (1987). This exemplary baculovirus expression vector contains the strong polyhedrin promoter of the Autographa californica nuclear polyhedrosis virus (AcMNPV) followed by the secretory signal peptide (leader) of the baculovirus gp67 polypeptide and convenient restriction sites such as BamH I, Xba I, and Asp 718. The polyadenylation site of the simian virus 40 (SV40) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate viable virus that expresses the cloned polynucleotide.

Other baculovirus vectors can be used in place of the vector above, e.g. pAc373, pVL941 and pAcIM1, as one skilled in the art would readily appreciate, as long as the construct provides appropriately located signals for transcription, translation, secretion and the like, including a signal peptide and an operably-linked AUG start codon as required (32).

The cDNA sequence encoding the mature polypeptide of interest lacking the AUG initiation codon (e.g., codons 18-337 of SEQ ID No 18) and the naturally associated nucleotide binding site, is amplified using PCR oligonucleotide primers corresponding to sequences upstream (5′) and downstream (3′) of the polynucleotide encoding the polypeptide of interest. Non-limiting examples include 5′ and 3′ primers having nucleotides corresponding to, or complementary to, a portion of the coding sequence of a polypeptide desired to be cloned, according to method steps known to those of skill in the art.

The amplified fragment is isolated from a 1% agarose gel using a commercially available kit (e.g., GENECLEAN, Qbiogene, Carlsbad, Calif.). The fragment is then digested with the appropriate restriction enzyme and again is purified on a 1% agarose gel. This fragment is designated herein “F1”.

The plasmid is digested with the corresponding restriction enzymes and optionally, can be dephosphorylated using calf intestinal alkaline phosphatase, using routine procedures known in the art. The DNA is then isolated from a 1% agarose gel using a commercially available kit. This vector DNA is designated herein “V1”.

Fragment F1 and the dephosphorylated plasmid V1 are ligated together with T4 DNA ligase. E. coli HB101 or other suitable E. coli hosts (e.g., XL-1 Blue, Stratagene, La Jolla, Calif.) cells are transformed with the ligation mixture and spread on culture plates. Bacteria are identified that contain the plasmid bearing the polynucleotide encoding the LP polypeptide of interest using the PCR method, in which one of the primers is that used to amplify the nucleic acid and a second primer is from well within the vector so that only those bacterial colonies containing the nucleic acid fragment encoding the LP polypeptide of interest will amplify the DNA. The sequence of the cloned fragment is confirmed by DNA sequencing. This plasmid is designated herein as pBacLP.

Five micrograms of the pBacLP plasmid is co-transfected with 1.0 μg of a commercially available linearized baculovirus DNA (BACULOGOLD™ baculovirus DNA, Pharmingen, San Diego, Calif.), using, for example, the lipofection method (33). 1 μg of BACULOGOLD™ virus DNA and 5 μg of the pBacLP plasmid are mixed in a sterile well of a microtiter plate containing 50 μL of serum-free Grace's medium (Invitrogen). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is rocked back and forth to mix the newly added solution. The plate is then incubated for 5 hours at 27° C. After 5 hours the transfection solution is removed from the plate and 1 ml of Grace's insect medium supplemented with 10% fetal calf serum is added. The plate is put back into an incubator and cultivation is continued at 27° C. for four days.

After four days the supernatant is collected and a plaque assay is performed. An agarose gel with “Blue Gal” (Invitrogen) is used to allow easy identification and isolation of gal-expressing clones, which produce blue-stained plaques. (A detailed description of a “plaque assay” of this type can also be found in the user's guide for insect cell culture and baculovirology distributed by Invitrogen). After appropriate incubation to allow for plaque growth, blue stained plaques are picked with a sterile micropipetor tip. The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and stored at 4° C.

To verify the expression of the LP polypeptide of interest, Sf9 cells are grown in Grace's medium supplemented with 10% heat-inactivated FBS. The cells are infected with the recombinant baculovirus expressing the polypeptide of interest at a multiplicity of infection (“MOI”) of about 2. Six hours later the medium is removed and is replaced with SF900 II medium minus methionine and cysteine (Invitrogen). If radiolabeled polypeptides are desired, 42 hours later, 5 mCi of³⁵S-methionine and 5 mCi ³⁵S-cysteine (Amersham) are added. The cells are further incubated for 16 hours and then they are harvested by centrifugation. The polypeptides in the supernatant as well as the intracellular polypeptides are analyzed by SDS-PAGE followed by autoradiography (if radiolabeled). Microsequencing of the amino acid sequence of the amino terminus of purified polypeptide can be used to determine the amino terminal sequence of the mature polypeptide and thus the cleavage point and length of the secretory signal peptide.

### Example 4

### Expression of Polypeptide in Mammalian Cells

A typical mammalian expression vector contains at least one promoter element (which mediates the initiation of transcription of mRNA), the polypeptide coding sequence, and signals required for the termination of transcription and polyadenylation of the transcript. Additional optional elements include enhancer(s), a Kozak sequence and an intervening sequence (intron) flanked by donor and acceptor sites for RNA splicing. Highly efficient transcription initiation can be achieved with the early and late promoters from SV40, the long terminal repeats (LTRs) from retroviruses (e.g., RSV, HTLV I, HIV) and the early promoter of the cytomegalovirus (CMV). However, cellular promoters can also be used (e.g., the human actin promoter). Suitable expression vectors for use in practicing the present invention include, but are not limited to, pIRES1neo, pRetro-Off, pRetro-On, PLXSN, or pLNCX (Clontech), pcDNA3.1 (+/−), pcDNA/Zeo (+/−) or pcDNA3.1/Hygro (+/−) (Invitrogen), pSVL and pMSG (Pharmacia, Uppsala, Sweden), pRSVcat (ATCC 37152), pSV2dhfr (ATCC 37146) and pBC12MI (ATCC 67109). Suitable mammalian host cells include, but are not limited to, human Hela 293 (ATCC CRL-1573), H9 (ATCC HTB-176), Jurkat cells (ATCC CRL-1990), mouse NIH3T3 (ATCC HB11601), C127 cells (ATCC CRL-1804), Cos 1, Cos 7 and CV 1, quail QC1-3 cells, mouse L cells (ATCC CCL-1) and Chinese hamster ovary (CHO) cells (ATCC CCL-61).

Alternatively, the nucleic acid encoding the polypeptide of interest is expressed in stable cell lines that contain the nucleic acid integrated into a host chromosome. The co-transfection of the nucleic acid encoding the polypeptide of interest along with a gene encoding a selectable marker such as DHRF(dihydrofolate reductase), GPT neomycin, or hygromycin allows the identification and isolation of the transfected cells.

The transfected gene can also be amplified to express large amounts of the encoded polypeptide. The DHFR marker is useful to develop cell lines that carry several hundred or even several thousand copies of the gene of interest. Another useful selection marker is the enzyme glutamine synthase (GS) (34, 35). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of polypeptides.

The expression vectors pC1 and pC4 contain the strong LTR promoter of the Rous Sarcoma Virus (36, 37). Multiple cloning sites (e.g., with the restriction enzyme cleavage sites BamH I, Xba I and Asp 718), facilitate the cloning of the gene of interest. The vectors contain, in addition to the 3′ intron, the polyadenylation and termination signal of the rat preproinsulin gene.

### Example 5

### Cloning and Expression of Polypeptides in COS Cells

The expression vector containing the nucleic acid encoding the polypeptide of interest, is made by cloning a cDNA encoding the polypeptide of interest into the expression vector pcDNAI/Amp or pcDNAIII (Invitrogen).

The expression vector pcDNAI/amp contains: (1) an E. coli origin of replication effective for propagation in E. coli and other prokaryotic cells; (2) an ampicillin resistance gene for selection of prokaryotic cells containing plasmid; (3) an SV40 origin of replication for propagation in eukaryotic cells; (4) a CMV promoter, a multiple cloning site polylinker, an SV40 intron; (5) several codons encoding a hemagglutinin fragment (i.e., an “HA” tag to facilitate purification) or HIS tag (see, e.g, Ausubel, supra) followed by a termination codon and polyadenylation signal arranged so that a cDNA can be conveniently placed under expression control of the CMV promoter and operably linked to the SV40 intron and the polyadenylation signal by means of restriction sites in the polylinker. The HA tag corresponds to an epitope derived from the influenza hemagglutinin polypeptide (38). The fusion of the HA tag to the target polypeptide allows easy detection and recovery of the recombinant polypeptide with an antibody that recognizes the HA epitope. pcDNAIII contains, in addition, the selectable neomycin marker.

A DNA fragment encoding the polypeptide of interest is cloned into the polylinker region of the vector so that recombinant polypeptide expression is directed by the CMV promoter to which it is operably linked. The plasmid construction strategy is as follows. The cDNA of a clone is amplified using primers that contain convenient restriction sites.

The PCR amplified DNA fragment and the vector, pcDNAI/Amp, are digested with suitable restriction enzyme(s) purified and ligated. The ligation mixture is transformed into E. coli cells (e.g., strain SURE, Stratagene, La Jolla, Calif.), and the transformed culture is plated on ampicillin LB media plates which then are incubated at 37° C. to allow growth of ampicillin resistant colonies. Plasmid DNA is isolated from resistant colonies and examined by restriction analysis or other means for the presence of the fragment encoding the polypeptide of interest.

One method for detection of an expressed fusion polypeptide is by radiolabeling and immunoprecipitation (39). Two days after transfection, the cells are labeled by incubation in medium containing ³⁵S-cysteine for 8 hours. The cells and the media are collected, and the cells are washed and lysed with detergent-containing RIPA buffer: 150 mM NaCl, 1% NP-40, 0.1% SDS (sodium dodecyl sulfate), 0.5% DOC (deoxycholate), 50 mM TRIS, pH 7.5. Proteins are purified from the cell lysate and from the culture media using a HA-specific monoclonal antibody. The purified polypeptides then are analyzed by SDS-PAGE and autoradiography. An expression product of the expected size is seen in the cell lysate, which is not seen in negative controls.

### Example 6

### Cloning and Expression of Polypeptide of Interest in CHO Cells

The vector pC4 is used for the expression of polypeptide of interest in CHO cells. Plasmid pC4 is a derivative of the plasmid pSV2-dhfr (ATCC Accession No. 37146). The plasmid contains the mouse DHFR gene under control of the SV40 early promoter. CHO cells or other cells lacking dihydrofolate activity that are transfected with these plasmids can be selected by growing the cells in a selective medium (alpha minus MEM, Invitrogen) supplemented with methotrexate. The amplification of the DHFR genes in cells resistant to methotrexate (MTX) has been well documented (40,41). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach can be used to develop cell lines carrying more than 1,000 copies of the amplified gene(s). Subsequently, when the methotrexate is withdrawn, cell lines are obtained which contain the amplified gene integrated into one or more chromosome(s) of the host cell.

Plasmid pC4 contains the LTR strong promoter of the Rous Sarcoma Virus (36) plus a fragment isolated from the enhancer of the immediate early gene of human CMV (37). Downstream of the promoter are BamH I, Xba I, and Asp 718 restriction enzyme cleavage sites that allow insertion of the genes. Downstream of these cloning sites the plasmid contains the 3′ intron and polyadenylation site of the rat preproinsulin gene. Other high efficiency promoters can also be used for expression, (e.g., human b-actin promoter, SV40 early or late promoters, or the LTR from other retroviruses). Clontech's Tet-Off and Tet-On gene expression systems and similar systems can be used to express the LP polypeptide of interest in a regulated way in mammalian cells. For the polyadenylation of the mRNA, polyadenylation signals, (e.g., from the human growth hormone or globin genes) can be used. Stable cell lines carrying a gene of interest integrated into the chromosomes can also be selected upon co-transfection with a gene expressing a selectable marker such as gpt, G418 or hygromycin. It is advantageous to use more than one selectable marker in the beginning, e.g., G418 plus methotrexate.

The plasmid pC4 is digested with appropriate restriction enzyme(s) and then dephosphorylated using calf intestinal phosphatase by procedures known in the art. The vector is then isolated from a 1% agarose gel.

The DNA sequence encoding the LP polypeptide of interst is amplified using PCR oligonucleotide primers corresponding to sequences 5′ and 3′ to the sequence of interest.

The amplified fragment is digested with suitable endonuclease(s) and then purified again on a 1% agarose gel. The isolated fragment and the dephosphorylated vector are then ligated with T4 DNA ligase. E. coli (e.g., HB101 or XL-1 Blue cells) are then transformed and bacteria are identified that contain the fragment inserted into plasmid pC4 using, for instance, using restriction enzyme analysis.

Chinese hamster ovary (CHO) cells lacking an active DHFR gene are used for transfection. Five micrograms of the expression plasmid pC4 is cotransfected with 0.5 μg of the plasmid pSV2-neo using e.g., lipofectin. The plasmid pSV2neo contains a dominant selectable marker, the neomycin resistance gene from Tn5 encoding an enzyme that confers resistance to a group of antibiotics including G418. The cells are seeded in alpha minus MEM supplemented with 1 μg/ml G418. After 2 days, the cells are trypsinized and seeded in hybridoma cloning plates (Greiner, Germany) in alpha minus MEM supplemented with 10, 25, or 50 ng/ml of methotrexate plus 1 μg/ml G418. After about 10-14 days clonal colonies are independently trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then independently transferred to a new well of a 6-well plate containing even higher concentrations of methotrexate (1 mM, 2 mM, 5 mM, 10 mM, 20 mM). The same procedure is repeated until clones are obtained which grow at a concentration of 100-200 mM methotrexate. Expression of the desired gene product is analyzed, for instance, by SDS-PAGE and Western blot or by reverse phase HPLC analysis.

### Example 7

### IL20 Polypeptide Purification

Cell culture media containing the expressed polypeptide of interest secreted from mammalian cell host (CHO DG44) transfected with the expression vector was concentrated in an Amicon ProFlux M12 tangential filtration system using an Amicon S3Y10 UF membrane. The concentrated media was passed over IMAC (Immobilized Metal-Affinity Chromatography (Pharmacia), 10 to 50 ml column) at a flow rate of 2 ml/min. The column was washed with buffer A (PBS, 1 mM potassium phosphate, 3 mM sodium phosphate), 0.15 M NaCl, pH 7.4 containing 50 mM Imidazol) until the absorbance returned to baseline. The bound polypeptides were eluted with a gradient from 100% Buffer A to 55% buffer A developed over 70 min. The gradient was then stepped to 100% Buffer B (Buffer A containing 0.5 M Imidazol) for 20 min. Fractions containing the purified polypeptide of interest were pooled and concentrated using an Ultrafree centrifugal filuter unit (Millipore, 10 kDa molecular weight cut off) to 15 ml. This material was passed over a Superdex 75 (Pharmacia, 26/60) sizing column equilibrated with PBS, (0.5 M NaCl, pH 7.4, at a flow rate of 3 ml/min. Fractions containing IL20 were analyzed by SDS-PAGE. The N-terminal sequence of IL20 was confirmed on the purified polypeptide.

### Example 8

### Cell Proliferation Assay

This assay demonstrates the level of inhibitory effect that a soluble receptor polypeptide (or combinations, fusions or variants thereof) of the invention has on the binding of a cytokine to its natural receptor. This assay may be performed in various combinations to test whether any soluble receptor polypeptide effectively inhibits the binding of a cytokine to its natural receptor and thereby inhibits or decreases signal transduction. The receptor subunits that comprise the natural receptor of a cytokine of interest are stably expressed in BAF-3 cells. The cytokine is added in the presence of varying amount of soluble receptor(s) to be tested and cell proliferation is monitored.

For example, IL20Rα and IL20Rβ may both be stably expressed in the same cell line that is then subsequently tested with a soluble receptor subunit, mixture, fusion or complex to determine if a cytokine that normally binds the full-length receptor being stably expressed in these cells (e.g., in this case, IL20, IL19 or IL24 or any combination thereof) is inhibited from binding to the receptor on the cell in the presence of the soluble receptor subunit, mixture, fusion or complex.

Exemplified here is the inhibition of IL20 binding to its normal receptor containing IL20Rα and IL20Rβ subunits. IL20, is a cytokine that binds to IL20Rα and IL20Rβ to form a trimeric complex. Formation of this ligand-receptor complex results in activation of the STAT3 signal transduction pathway. It is well established that ligand binding to Class 2 cytokine receptors (such as IL20Rα/IL20Rβ) leads to activation of JAK kinases and subsequent phosphorylation of STAT proteins, the latter then undergo nuclear translocation where they transcriptionally activate target genes that have a promoter responsive to the STAT protein. In addition, many cytokines also activate the MAP kinase pathway which is an alternate means of leading to transcriptional activation of target genes. Since the above-mentioned pathways are known to exist in the IL3 dependent BAF-3 cell line and transfection of this cell line with another receptor (e.g., G-CSF(45)) converts them to dependence on G-CSF, the stable expression of IL20Rα and IL20Rβ in BAF-3 cells converts them to being dependent upon IL20 as demonstrated herein. Expression of a different cytokine receptor in BAF-3 cells converts them to being dependent upon the cytokine that normally binds that receptor and can be used in a cell proliferase assay to demonstrate responsiveness to the cytokine or inhibition of the cytokine by a soluble receptor of interest.

An IL3-dependent murine pre-B cell line BaF3 (43) grown in RPMI 1640 media supplemented with 10% FBS and 0.5 ng/ml IL3 (R&D Biosystems, Mpls, Minn.) were centrifuged at 1000 RPM in a Jouan CR422 centrifuge and resuspended in PBS (10,30) at 2.2×10⁷ cells/ml. Then 220 μl cells were transfected by electroporation with an expression vector containing the coding sequence of one of the full-length cytokine receptor subunits (e.g., IL20Rα). The transfected BaF3 cells are resuspended in 20 ml RPMI 1640 media supplemented with 10% FBS and 0.5 ng/ml IL3 in a T75 flask. After 48 hours, selection was initiated by adding to the media an antibiotic (G418 at 600 μg/ml, Gibco) to isolate the antibiotic-resistant transfectants. Pools of the transfectants IL20Rα are then further transfected with an expression vector containing the coding sequence of a second full-length cyokine receptor subunit (e.g., IL20Rβ) using the same approach and selected in media containing a cytokine (e.g. IL20) to which the cells expressing both subunits are now dependent. (Alternatively, cells could be cotransfected with both subunits at the same time). Single cell cloning was performed by either limited dilution or through the use of a cell sorter. Clones were maintained in RPMI 1640 media supplemented with 20% FBS, 200 ng/ml IL20 (or other cytokine may be used for other types of receptors expressed). Expression of both receptor subunits is further confirmed by any of a variety of suitable methods including reverse transcriptase-PCR, Northern blot analysis and in situ hybridization.

For the bioassay, the cells maintained in RPMI 1650 supplemented with 10% FBS and 200 ng/ml of the appropriate cytokine are washed in PBS and resuspended in phenol red-free RPMI 1640 media supplemented with 5% FBS at a concentration of 0.4×10⁶ cells/ml. To each well of a 96 well plate is added 50 μl of cells and 50 μl of IL20 at a concentration of 4 ng/ml plus or minus increasing concentrations of the purified IL20Rαs and IL20Rβs or a combination of both. After 20 hours, 37.5 μl of CellTiter 96 Aqueous solution (Promega Corp., Madison, Wis.) is added and after a further three hour incubation, the plate is read on an ELISA plate reader at 490 nM to determine the number of metabolically active cells. The data from these experiments is shown below in Table 4. Each point represents the mean of 4 data points with the standard deviation (SD) indicated. Values are expressed as the percent inhibition of the response observed with 4 ng/ml of IL20 subtracted from the base line (0 ng/ml IL20).

TABLE 4a
Standard Curve with IL20

| [IL20] | | |
|---|---|---|
| ng/ml | Mean | SD |
| 0 | 0.274 | 0.013 |
| 0.01 | 0.298 | 0.022 |
| 0.1 | 0.313 | 0.028 |
| 0.3 | 0.323 | 0.045 |
| 1 | 0.316 | 0.015 |
| 3 | 0.325 | 0.025 |
| 10 | 0.482 | 0.022 |
| 30 | 0.555 | 0.025 |
| 100 | 0.716 | 0.034 |
| 300 | 0.772 | 0.079 |
| 1000 | 0.858 | 0.047 |
| 3000 | 0.933 | 0.061 |
| 10000 | 0.872 | 0.019 |

TABLE 4b
Soluble Receptor Cell Proliferation relative to control

| μg/ml | | | | | IL20Rβs + | |
|---|---|---|---|---|---|---|
| added: | IL20Rβs | SD | IL20Rαs | SD | IL20Rαs | SD |
| 0.0 | 100 | 0 | 100 | 0 | 100 | 0 |
| 0.06 | 101.4 | 7.9 | 99.3 | 15.7 | ND | ND |
| 0.12 | 101.4 | 7.3 | 95 | 8.3 | 103.2 | 11.4 |
| 0.3 | 91.6 | 10.7 | 89.4 | 9.7 | 97.4 | 7.1 |
| 0.6 | 78.9 | 12.2 | 78.4 | 6.8 | 75.8 | 3.0 |
| 1.2 | 85.2 | 2.1 | 84.6 | 6.7 | 69.4 | 11.6 |
| 3 | 77.3 | 9.8 | 95.1 | 3.2 | 77.3 | 13.3 |
| 6 | 69.5 | 3.7 | 93.3 | 10.7 | 55.9 | 6.4 |
| 12 | 42.3 | 14.7 | 95.5 | 6.9 | 27.9 | 8.8 |

The numbers reflect the amount of cell proliferation relative to control without any inhibitor. 100—the value above is equal to the percent inhibition.

These data demonstrate that IL20Rβs alone inhibits IL20 induced cell proliferation while IL20Rαs alone does not inhibit IL20 induced cell proliferation. However, IL20Rαs enhances the inhibition of IL20 induced cell proliferation observed with IL20Rβs alone. For example, the value of 72.1% inhibition with 12 μg/ml of IL20Rβs+IL20Rαs is from 6 μg/ml each of IL20Rβs and IL20Rαs. This is greater than the 30.5% inhibition observed with 6 μg/ml IL20Rβs alone.

An additional assay using IL20Rβs, exactly as described above with the exception that the IL20 concentration was 6.6 ng/ml, was performed. Four data points were read at each point and the mean and standard deviation are shown in Table 5 hereinbelow.

TABLE 5

| [IL20Rβs] | | |
|---|---|---|
| (μg/ml) | Mean | SD |
| 0.00 | 100.0 | 0.00 |
| 0.06 | 65.93 | 11.95 |
| 0.19 | 52.43 | 10.38 |
| 0.57 | 49.44 | 3.73 |
| 1.88 | 40.37 | 3.94 |
| 5.66 | 30.67 | 5.68 |
| 11.33 | 7.92 | 5.93 |

It is readily apparent to those of ordinary skill in the art in light of the teachings of this invention that certain changes and modifications may be made thereto without departing from the spirit or scope of the appended claims. It is to be understood that no limitation with respect to the specific examples presented is intended or should be inferred. The disclosure is intended to cover by the appended claims modifications as fall within the scope of the claims.

Reference Listing

1. Moore, K., et al. Annu. Rev. Immunol. 2001. 19:683-765.

2. Blumberg, H., et al. Cell. 2001. 104:9-19.

3. Su, Z., et al. Proc. Natl. Acad. Sci. 1998. 95:14400-14405.

4. xie, M., et al. J. Biol. Chem. 2000. 275:31335-31339.

5. Dumoutier, L., et al. J. Immunology. 2001. 167:3545-3549.

6. Grantham, et al., Nuc. Acids Res. 1980. 8:1893-1912.

7. Haas, et al. Curr. Biol. 1996.6:315-324.

8. Ikemura, J. Mol. Biol. 1982. 158:573-597.

9. Altschul et al., Methods in Enzymology 1996. 266:460-480.

10. Ausubel et al., Current Protocols in Molecular Biology, Wiley Interscience Publishers, 1995 and supplements.

11. Smith and Waterman. Adv. Appl. Math. 1981. 2:482.

12. Needlman and Wunsch. J. Mol. Biol. 1970. 48:443.

13. Pearson and Lipman Proc. Natl. Acad. Sci. 1988. 85:2444.

14. Brutlag, et al. Comp. App. Biosci. 1990. 6:237-245.

15. Bowie, et al. Science. 1990. 247:1306-1310.

16. Cunningham and Wella. Science. 1989. 244:1081-1085.

17. Cleland, et al. Crit. Rev. Ther. Drug Carrier Systems. 1993. 10:307-377.

18. Creighton, Proteins—Structure and Molecular Properties. 2^{nd} Ed. W. H. Freeman and Co. New York, 1993.

19. Johnson. Posttranslational Covalent Modification of Proteins. Academic Press, New Yor, pp 1-12. 1983.

20. Seifter, et al. Meth. Enzymol. 1990. 182:626-646.

21. Rattan et al. Ann. NY Acad. Sci. 1992. 663:48-62.

22. Aplin and Wriston. CRC Crit. Rev. Biochem. 1981, pp. 259-306.

23. Thotakura, et al. Meth. Enzymol. 1987. 138:350-359.

24. Pouwels, et al. Cloning Vectors: A Laboratory Manula. 1985 and Supplements, Elsevier, N.Y.

25. Chasin, Proc. Natl. Acad. Sci. 1980. 77:4216-4220.

26. Stinchcomb, et al. Nature. 1979. 282:39-43.

27. Butler, M. ed. Mammalian Cell Biotechnology. IRL Press, 1991.

28. Deutscher, Methods in Enzymology. 1990. 182:83-89.

29. Dzau, et al. Trends in Biotechnology. 1993. 11:205-210.

30. Sambrook, et al, Molecular Cloning, A Laboratory Manual. 1989. Cold Spring Harbor Press, Ny.

31. Pear, et al. Proc. Natl. Acad. Sci. 1993. 90:8392.

32. Luckow, et al., Virology 170:31-39.

33. Felgner, et al. Proc. Natl. Acad. Sci. 1987. 84:7413.

34. Murphy, et al., Biochem. J. 1991. 227:277-279.

35. Bebbington, et al. BioTechnology 1992. 10:169-175.

36. Cullen, et al. Mol. Cell. Biol. 1985. 5:438-447.

37. Boshart, et al. Cell. 1985. 41:521-530.

38. Wilson, et al. Cell. 1984. 37:767-778.

39. Harlow, et al. Antibodies: A Laboratory Manual, 2^{nd} Ed., 1988. CSHLP. Cold Spring Harbor, N.Y.

40. Hamlin and Ma. Biochem. Et Biophys. Acta. 1990. 1097:107-143.

41. Page and Sydenham. BioTechnology 1991. 9:64-68.

42. Xu, W. et al. Proc. Natl. Acad. Sci. 2001. 98:9511-9516.

43. Palacios, and Steinmetz. Cell. 1985. 41:727-734.

44. Gallagher, et al. Genes and Immunity. 2000. 1:442-450.

45. Bashey, et al. Blood. 1994. 83:949-957.

1. A method for treating or preventing a disease or disorder selected from the group consisting of inflammation, immune system, cardiovascular, hematopoietic and regulation of cellular proliferation in a mammal comprising the administration to said mammal in need of such treatment a pharmaceutical composition comprising a therapeutically effective amount of a polypeptide comprising an amino acid sequence selected from. the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9.

2. A method for down-regulating the activity of a cytokine selected from the group consisting of IL19, IL-20, and IL24 in a mammal comprising the administration to said mammal in need of such cytokine down-regulation a pharmaceutical composition comprising a therapeutically effective amount of a polypeptide comprising an amino acid sequence selected from the group consisting of, SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, and SEQ ID NO: 9.

3. A method for treating or preventing a disease or disorder selected from the group consisting of inflammation, immune system, cardiovascular, hematopoietic and regulation of cellular proliferation in a mammal comprising the administration to said mammal in need of such treatment a pharmaceutical composition comprising a therapeutically effective amount of a variant of a polypeptide comprising an amino acid sequence at least 95% identical to an amino acid sequence selected from the group consisting of, SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, and SEQ ID NO: 9.

4. The method of any one of claims 1, 2 or 3, wherein the pharmaceutical composition further comprises a pharmaceutically acceptable carrier, diluent or excipient.

5. The method of any one of claims 1, 2, 3 or 4 wherein the disease or disorder is an inflammation disease or disorder.

6. The method of any one of claims 1, 2, 3 or 4 wherein the disease or disorder is a hematopoietic disease or disorder.

7. A fusion protein having a first polypeptide and a second polypeptide wherein the first polypeptide comprises an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9 and the second polypeptide comprises an amino acid sequence selected from the group consisting of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, and SEQ ID NO: 9.

8. An isolated polypeptide comprising the amino acid sequence shown in SEQ ID NO: 4.

9. (Cancelled)

10. A pharmaceutical composition comprising an inflammation-decreasing amount of an isolated polypeptide comprising an amino acid sequence selected from the group consisting of
a) SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9
b) a variant at least 95% identical to that of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, and SEQ ID NO: 9, and
c) a fusion protein having a first polypeptide and a second polypeptide wherein the first polypeptide comprises an amino acid sequence selected from the group consisting of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, and SEQ ID NO: 9 and the second polypeptide comprises an amino acid sequence selected from the group consisting of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, and SEQ ID NO: 9,
and further comprising a pharmaceutically acceptable carrier, diluent or excipient.

11. A pharmaceutical composition comprising a hematopoietic disease or disorder treating amount of an isolated polypeptide comprising an amino acid sequence selected from the group consisting of
a) SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9
b) a variant at least 95% identical to that of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, and SEQ ID NO: 9 and
c) a fusion protein having a first polypeptide and a second polypeptide wherein the first polypeptide comprises an amino acid sequence selected from the group consisting of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, and SEQ ID NO: 9 and the second polypeptide comprises an amino acid sequence selected from the group consisting of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, and SEQ ID NO: 9,
and further comprising a pharmaceutically acceptable carrier, diluent or excipient.

12. A pharmaceutical composition comprising an immune system, or cardiovascular system, disease or disorder treating amount of an isolated polypeptide comprising an amino acid sequence selected from the group consisting of
a) SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9
b) a variant at least 95% identical to that of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9, and
c) a fusion protein having a first polypeptide and a second polypeptide wherein the first polypeptide comprises an amino acid sequence selected from the group consisting of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, and SEQ ID NO: 9 and the second polypeptide comprises an amino acid sequence selected from the group consisting of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, and SEQ ID NO: 9,
and further comprising a pharmaceutically acceptable carrier, diluent or excipient.
